SEQUENCE LISTING (53 sequences)

SEQ ID NO: 1 - DNA, 23 bp, Artificial Sequence
  Synthetic
agaaaaagagcgggagcgggcga

SEQ ID NO: 2 - DNA, 23 bp, Artificial Sequence
  Synthetic
caggggggagcgaagggaaacag

SEQ ID NO: 3 - DNA, 23 bp, Artificial Sequence
  Synthetic
aaggggggtgggagaggagagaa

SEQ ID NO: 4 - DNA, 53 bp, Artificial Sequence
  Synthetic
aggaagagaaaccaagatagaatctcacaatatctgatatattagattgactc

SEQ ID NO: 5 - DNA, 52 bp, Artificial Sequence
  Synthetic
gagatatttttgtcacaatgagaggtggtgagtgctactggcatctagtgtg

SEQ ID NO: 6 - DNA, 52 bp, Artificial Sequence
  Synthetic
aaatggaaagaggtaggagatcagagcagaagggtgtgagaaaaaggaaatc

SEQ ID NO: 7 - DNA, 52 bp, Artificial Sequence
  Synthetic
gtattttgaaataacatttttttgaacaagctattgtagtagaacagacata

SEQ ID NO: 8 - DNA, 53 bp, Artificial Sequence
  Synthetic
cccttggaagcatagactgatcttcacacagggaatgtaaaaaatttaccatg

SEQ ID NO: 9 - DNA, 52 bp, Artificial Sequence
  Synthetic
cccgtcagtgtcaggaagaagaaaccggagtactagtaccccatctccagct

SEQ ID NO: 10 - DNA, 52 bp, Artificial Sequence
  Synthetic
aaatctggattgtattactctggctctgagattctacattctagcctaggta

SEQ ID NO: 11 - DNA, 52 bp, Artificial Sequence
  Synthetic
taaaaacttttatgagtagactcattaaagtttttcttgaaagaatggaaag

SEQ ID NO: 12 - DNA, 51 bp, Artificial Sequence
  Synthetic
atgctaacagtattcttatttgtgaagtgaattgatttggagaggctggcg

SEQ ID NO: 13 - DNA, 50 bp, Artificial Sequence
  Synthetic
gtaaagactttagagcaaagactgcagggtagtggtcgtgggccagattc

SEQ ID NO: 14 - DNA, 50 bp, Artificial Sequence
  Synthetic
ttgcaacccagccaggtgtgtgcgtactcagggcagcactgacataccag

SEQ ID NO: 15 - DNA, 48 bp, Artificial Sequence
  Synthetic
atgtgaagcagctgtagaagaaaagtttgaaactagcagataatggtt

SEQ ID NO: 16 - DNA, 52 bp, Artificial Sequence
  Synthetic
gcagaggctggccaaggggcgaagcgtaagaaaagtatggaacttcacttca

SEQ ID NO: 17 - DNA, 46 bp, Artificial Sequence
  Synthetic
aaaagaagttaaaatataaaatgatcatttcactgaacttttgtca

SEQ ID NO: 18 - DNA, 46 bp, Artificial Sequence
  Synthetic
gaatacaggtgccacaactgagaaacaaggtaaggaaaagcaggat

SEQ ID NO: 19 - DNA, 50 bp, Artificial Sequence
  Synthetic
aaaagaatacaggtgccacaactgaaaaacaaggtaaggaaaagcaggat

SEQ ID NO: 20 - DNA, 48 bp, Artificial Sequence
  Synthetic
gaagactgacattgtcagggagaatacagacaatattagtaagtggcc

SEQ ID NO: 21 - DNA, 50 bp, Artificial Sequence
  Synthetic
gatcttttatgcacctcgtcccaagcccatgagggcaagagctgtataag

SEQ ID NO: 22 - DNA, 47 bp, Artificial Sequence
  Synthetic
agatcctgaccatattttacaaatttattatactcagtcctgatatt

SEQ ID NO: 23 - DNA, 55 bp, Artificial Sequence
  Synthetic
taggaattccagctatcttagatattcctatgcccagtgggggagttttgcaaag

SEQ ID NO: 24 - DNA, 53 bp, Artificial Sequence
  Synthetic
actaactgggcgtaagcttggaatgtagtatctttgaaattgcaggtcaaagc

SEQ ID NO: 25 - DNA, 52 bp, Artificial Sequence
  Synthetic
ttgttttgatattcatattttattgtgttacacacaagtctgcaggaacaca

SEQ ID NO: 26 - DNA, 52 bp, Artificial Sequence
  Synthetic
ctctgctgtgggttcaagcacccctgaggcttctgaggagtcagtcaaaata

SEQ ID NO: 27 - DNA, 52 bp, Artificial Sequence
  Synthetic
caggtttagaggagccttgggtcacacagttggcaattcagtggcccaggac

SEQ ID NO: 28 - DNA, 52 bp, Artificial Sequence
  Synthetic
gtgagccctcctagtgattgtgtccggacagcctggggaactggcaggagac

SEQ ID NO: 29 - DNA, 52 bp, Artificial Sequence
  Synthetic
gtcacattttgaagcttacaaagggttgccctctattctgttcagtctagtg

SEQ ID NO: 30 - DNA, 49 bp, Artificial Sequence
  Synthetic
tgacatctatgagaggggattaaaggccctaccctgttgccctcattta

SEQ ID NO: 31 - DNA, 52 bp, Artificial Sequence
  Synthetic
gagatgctgttcttttgtgtcctcagcgtttgcctaatccttggctattttt

SEQ ID NO: 32 - DNA, 52 bp, Artificial Sequence
  Synthetic
cttgactgtgccaggttggaaaatttactggtttatgaagaaaaggcatttt

SEQ ID NO: 33 - DNA, 52 bp, Artificial Sequence
  Synthetic
tgaggctctcaggggaaacccagatgggcagctgggcctggctgggaagggc

SEQ ID NO: 34 - DNA, 50 bp, Artificial Sequence
  Synthetic
ggcagactggagtatataagagaaaaacattgctttgtgtgaggcatggt

SEQ ID NO: 35 - DNA, 51 bp, Artificial Sequence
  Synthetic
gttaacctctcccagacttcagcagtgtttacagaagatgtttcacatcca

SEQ ID NO: 36 - DNA, 52 bp, Artificial Sequence
  Synthetic
acaagaagatgagaattgaaactccgcgttctcatcggagagaaaatcagac

SEQ ID NO: 37 - DNA, 56 bp, Artificial Sequence
  Synthetic
gattatgtgcaaaaacacagtcatagcaaaagtttaacttttgagaaaaccccttt

SEQ ID NO: 38 - DNA, 50 bp, Artificial Sequence
  Synthetic
cacggtaaaaatacaaggacagtgtgtgcagcagaatggccaggcagacc

SEQ ID NO: 39 - DNA, 53 bp, Artificial Sequence
  Synthetic
aaaagataataccacacaaacagtaatcatacgaaagttggggtggccatatt

SEQ ID NO: 40 - DNA, 51 bp, Artificial Sequence
  Synthetic
tgtaaaaaagacaaagttgttgggacacgtaagtatggcgtctgaaactgc

SEQ ID NO: 41 - DNA, 51 bp, Artificial Sequence
  Synthetic
agtgggcggctaaatacatcaggaaccgcatcatccagtttaacccagggc

SEQ ID NO: 42 - DNA, 55 bp, Artificial Sequence
  Synthetic
ggcacactcttattgtattcctactaaatgtcttttccacagcgctgatagagtc

SEQ ID NO: 43 - DNA, 54 bp, Artificial Sequence
  Synthetic
ctttctggaagaactgaagtcttctccttcctccgccacagataccccgcccag

SEQ ID NO: 44 - DNA, 53 bp, Artificial Sequence
  Synthetic
agttactagagcactgcttaccatgaaggcttacatatgagtaacgaagaaac

SEQ ID NO: 45 - DNA, 46 bp, Artificial Sequence
  Synthetic
tgggaggactgttagccagttaagctgtgattttaagctggagtaa

SEQ ID NO: 46 - DNA, 57 bp, Artificial Sequence
  Synthetic
gaagagtgttagcctcttggagtaacctacagatgttaacaacagcacaactagact

SEQ ID NO: 47 - DNA, 56 bp, Artificial Sequence
  Synthetic
caaagtctccttcgagagctagatgaaactttggaaaaacctcagtgtatctccgc

SEQ ID NO: 48 - DNA, 50 bp, Artificial Sequence
  Synthetic
tacattagctggttagtcttccccaaatgaggagatgcacactgcaagta

SEQ ID NO: 49 - DNA, 53 bp, Artificial Sequence
  Synthetic
gggagtggcgggaaatggggggcatcaccatgcctgccgtcgggttcctgcgc

SEQ ID NO: 50 - DNA, 53 bp, Artificial Sequence
  Synthetic
caacagtgacgaggagataaccaggtgaggtgtgagaggatgcagaagtgacc

SEQ ID NO: 51 - DNA, 57 bp, Artificial Sequence
  Synthetic
taaactatcttcttgtccattgagatgcacagctcccactccctatggatgtgtttg

SEQ ID NO: 52 - DNA, 57 bp, Artificial Sequence
  Synthetic
gaaaatagaggctctccaagggttccgttaaaggagagaataatcagaggtgaagaa

SEQ ID NO: 53 - DNA, 58 bp, Artificial Sequence
  Synthetic
gttaaccaaatgctctttatgatttattattattctatttctgggcagtttcactgcc

(19) United States
(12) Patent Application Publication
(10) Pub. No.: US 2013/0012497 A1
(43) Pub. Date: Jan. 10, 2013

(54) METHODS FOR DETECTING ADHD SUSCEPTIBILITY AND PREDICTING SEVERITY AND LONG TERM OUTCOME OF ADHD SYMPTOMS

(75) Inventors: Maximilian Muenke, Bala Cynwyd, PA (US); Mauricio Arcos-Burgos, Canberra (AU); Maria Teresa Acosta, Bethesda, MD (US)

(73) Assignee: The United States of America, as represented by the Secretary, Department of Health and Human Ser., Bethesda, MD (US)

(21) Appl. No.: 13/475,342

(22) Filed: May 18, 2012

Related U.S. Application Data
(60) Provisional application No. 61/505,864, filed on Jul. 8, 2011.

(51) Int. Cl. C40B 30/04; A61K 31/519; A61K 31/165; C12Q 1/68; A61K 31/55; A61K 31/135; A61P 25/26; A61P 25/24; A61K 31/4168; A61K 31/137

(52) U.S. Cl. 514/217; 514/398; 514/259.41; 514/618; 514/654; 514/656; 506/9; 435/6.11

(57) ABSTRACT

The invention provides methods of determining an altered susceptibility to develop ADHD, methods of predicting or determining the severity of symptoms of ADHD and associated disorders, and methods of treatment based on the presence or absence of one or more alleles of single nucleotide polymorphism (SNP) markers.

### RELATED APPLICATIONS

This patent application claims the benefit of U.S. Provisional Patent Application No. 61/505,864, filed Jul. 8, 2011, which is incorporated by reference herein in its entirety.

### INCORPORATION-BY-REFERENCE OF MATERIAL SUBMITTED ELECTRONICALLY

Incorporated by reference in its entirety herein is a computer-readable nucleotide sequence listing submitted concurrently herewith and identified as follows: One 9,991 Byte ASCII (Text) file named “710265ST25.TXT,” created on Apr. 17, 2012.

### BACKGROUND OF THE INVENTION

Attention-Deficit/Hyperactivity Disorder (ADHD) is one of the most common behavioral disorders of childhood, affecting approximately 10% of children and adolescents in the United States and 8-12% of children worldwide. Visser et al., Mortality and Morbidity Weekly Report, 59: 1439-1443 (2010); Biederman, Lancet, 366: 237-248 (2005). ADHD is characterized by elevated levels of inattention, excessive motor activity, and impulsive behaviors that can significantly impair social and academic performance. Affected individuals are at increased risk for poor educational achievement, low income, underemployment, legal difficulties, and impaired social relationships. Faraone et al., J. Acad. Child Adolesc. Psychiatry, 35: 1449-1459 (1996).

The severity of ADHD symptoms, including the severity of ADHD-associated disorders, is a major predictor of long-term ADHD outcome. Barry et al., Journal of School Psychology., 40(3): 259-83 (2002); Riley et al., Eur. Child Adolesc. Psychiatry, Suppl 1: 138-45 (2006), Upadhyaya et al., Am. J. Addict., 17(3): 195-8 (2008); and Norvilitis et al., J. Learn. Disabil., 43(1): 86-94 (2010). A traditional approach to evaluating the severity of ADHD and associated disorders includes using the diagnostic criteria for ADHD as defined in the American Psychiatric Association, Diagnostic and Statistical Manual of Mental Disorders 4th Ed., rev. (2000) (DSM-IV). This approach may fail to identify individuals who do not fit the overall numeric criteria for ADHD diagnosis according to DSM-IV and it may overlook individuals with a severe presentations of one or more symptoms of ADHD and associated disorders. Severe ADHD symptoms, including ADHD-associated disorders, can adversely impact functional outcome for such individuals who, though undiagnosed according to DSM-IV, may benefit from clinical intervention.

Genetic factors are implicated in the etiology of ADHD. Biederman, Lancet, 366: 237-248 (2005). For instance, a haplotype in the genetic locus 4q13.2, i.e., the latrophilin 3 gene (LPHN3), has been reported to increase the risk of developing ADHD. Arcos-Burgos, et al., Mol. Psychiatry., 15(11): 1053-1066 (2010); Ribases et al., Genes Brain Behav., 10(2): 149-157 (2010). Other genetic loci that have been independently linked to ADHD include 5q, 8q, 11q, and 17p, and minimal critical regions (MCR) for each one of these regions have been reported. Arcos-Burgos et al., Am. J. Hum. Genet., 75(6): 998-1014 (2004). However, studies have not yet determined whether these genetic loci interact in a diagnostically or clinically relevant manner in the development of ADHD and its manifestations.

A number of difficulties must be overcome in mapping disorders such as ADHD which are thought to have complex origins, low penetrance, heterogeneity, pleiotropy, and epistasis. Identifying and accounting for these potential complexities are of great importance when searching for the specific genetic variations that underlie the susceptibility for and clinical presentation of ADHD.

### BRIEF SUMMARY OF THE INVENTION

The invention provides methods which take into account the complexity of underlying interacting genetic factors in determining and predicting an individual's susceptibility to develop ADHD. The invention is based, in part, on the discovery of interactions between genetic biomarkers (“markers”) located on 4q13.2 (i.e., LPHN3) and interacting markers located on other chromosome regions which together indicate an increased susceptibility to develop Attention Deficit Hyperactivity Disorder (ADHD). The invention also provides methods which reflect the complexity of underlying genetic factors in assessing the severity of symptoms of ADHD and ADHD-associated disorders in an individual. In this regard, the invention is based, in part, on the discovery that markers on chromosomes 4q, 5q, 5p, 11q, and 12q and combinations of these markers can be used to determine or predict ADHD severity, i.e., the severity of on or more symptoms of ADHD and associated disorders.

As used herein, the term “marker” refers to a single nucleotide polymorphism (SNP). A susceptibility marker refers to a SNP allele that indicates an increased risk for ADHD and/or a more severe presentation of at least one symptom of ADHD or an associated disorder, as compared to a subject without the susceptibility marker. A protective marker refers to a SNP allele that indicates a decreased risk for ADHD and/or decreased likelihood of having or developing one or more severe symptoms of ADHD or an associated disorder, as compared to a subject without the protective marker.

In one embodiment, the invention provides a method of determining whether a subject has an increased susceptibility to develop ADHD. The method includes providing a sample from the subject and analyzing the sample for (i) one or more ADHD susceptibility markers in LPHN3 gene region spanning positions 61,650,000-62,650,000 on chromosome 4q13.2 and (ii) one or more interacting ADHD susceptibility markers located on a second, different chromosome region. The presence of at least one ADHD susceptibility marker in LPHN3 and the presence of at least one interacting ADHD susceptibility marker indicate that the subject has an increased susceptibility to develop ADHD. In one example, the one or more interacting ADHD susceptibility markers are located in the region spanning positions 112,250,000-112,900,000 of chromosome 11q.

In another embodiment, the invention provides a method of predicting or determining the severity of one or more symptoms of Attention Deficit Hyperactivity Disorder (ADHD) or an associated disorder in a subject. The method includes providing a sample from the subject and analyzing the sample for one or more ADHD susceptibility or protective markers linked to one (or more) of the following genes: LPHN3 on chromosome 4q, NDFIP1 on chromosome 5q, GNPDA1 on chromosome 5q, SLC25A48 on chromosome 5q, SLC6A3 on chromosome 5p, DRD2 on chromosome 11q, NCAM1 on chromosome 11q, TTC12 on chromosome 11q, the intergenic region between NCAM1 and TTC12 on chromosome 11q, or TPH2 on chromosome 12q. The presence of the one or more ADHD susceptibility markers indicates or predicts the severity of one or more symptoms of ADHD or an associated disorder. In one example, the method includes analyzing the sample for (i) one or more ADHD susceptibility markers in LPHN3 located in the region spanning position 61,650,000-62,650,000 on chromosome 4q and (ii) one or more ADHD susceptibility markers located in the region spanning positions 112,250,000-112,900,000 of chromosome 11q.

Additionally, the invention provides methods for monitoring or treating a subject. In an embodiment, a method includes determining whether the subject has an increased susceptibility to develop ADHD as disclosed herein. If the subject has an increased susceptibility to develop ADHD, the method includes monitoring the subject for development or worsening of ADHD symptoms and/or treating the subject for ADHD. If the subject does not have an increased susceptibility for developing ADHD, the method can include providing treatment for a condition other than ADHD or, alternatively, providing no treatment as appropriate.

Another method of monitoring or treating a subject includes predicting or determining the severity of one or more symptoms of ADHD or an associated disorder in the subject as disclosed herein. If the subject is predicted or determined to have a severe form of ADHD, i.e., more severe presentation of symptoms of ADHD or an associated disorder, the method includes providing the subject with appropriate treatment for the one or more severe symptoms. If the subject is predicted or determined not to have a severe form of one or more symptoms of ADHD or an associated disorder, the method can include tailoring treatment for the subject. The tailored treatment can omit unnecessary therapeutic regimens intended to treat the severe symptoms associated with ADHD, which the subject is predicted or determined to be without.

### BRIEF DESCRIPTION OF THE DRAWINGS

FIG. 1 shows the sequences of ADHD susceptibility marker alleles of rs7678046, rs1901223 rs6813183, and rs1355368 in a susceptibility haplotype of the LPHN3 gene (SEQ ID NO:1). FIG. 1 also shows the corresponding sequences of an ADHD protective haplotype of LPHN3 (SEQ ID NO:2) and consensus human genome sequence (SEQ ID NO:3).

FIG. 2 shows the sequence of ADHD susceptibility markers (SEQ ID NOs: 4-13) in the region of chromosome 4q13.2 that includes the LPHN3 gene. The single nucleotide polymorphism site for each marker is bracketed and underlined for reference.

FIG. 3 shows the sequence of an ADHD protective marker (SEQ ID NO. 14) and ADHD susceptibility markers (SEQ ID NOs: 15-23) in the region of chromosome 4q13.2 that includes the LPHN3 gene. The single nucleotide polymorphism site for each marker is bracketed and underlined for reference.

FIG. 4 shows the sequence of ADHD susceptibility markers (SEQ ID NOs: 24-34) and ADHD protective markers (SEQ ID NOs: 35-36 and 49) in the region of chromosome 11q that includes the DRD2, NCAM1, and TTC12 genes. The single nucleotide polymorphism site for each marker is bracketed and underlined for reference.

FIG. 5 shows the sequence of susceptibility markers on chromosomes 11q (SEQ ID NOs: 50, 52, and 53), 5q (SEQ ID NOs: 38, 39, and 41) and 17p (SEQ ID NOs: 40-41) and protective markers on chromosomes 11q (SEQ ID NO:51), 5q (SEQ ID NOs: 37 and 40), and 12q (SEQ ID NO. 42). The single nucleotide polymorphism site for each marker is bracketed and underlined for reference.

FIG. 6A is a graph that demonstrates the results of a correlation subset analysis in 134 nuclear families (primarily derived from multigenerational extended pedigrees) for linkage to chromosome 11q. The graph plots chromosomal position in chromosome 11q (x-axis) against non-parametric statistic score (y-axis) position. FIG. 6A demonstrates an increase in the nonparamateric linkage statistic score from 0.55 to 3.2 in families linked to 4q (n=12) and an increase from 0.55 to 3.88 (n=11) when conditioning on families linked to 17p.

FIG. 6B is a graph showing the maximal nonparametric logarithm of the odds (LOD) score for the interaction between 4q and 11q (6B, n=12). The maximal nonparametric score of 6.08 (P<0.00000001) is located at 111.1 cM on 11q at SNP marker rs1293344 and 91.3 cM on 4q in the vicinity of rs1038426.

FIG. 6C is a graph depicting the maximal nonparametric LOD score for the interaction between 11q and 17p. The maximal nonparametric score of 5.51 (P<0.000001) is located at 111.1 cM on 11q at SNP marker rs1293344 and 12.75 cM on 17p in the vicinity of rs9227.

FIG. 7 is a Venn diagram showing the linkage percentage of markers on chromosomes 4q, 11q and 17p among twenty-three families with such markers. Fewer families are linked to 11q alone compared to 4q and 17p. 47% of the families demonstrated linkage to more than one region and 4% demonstrated linkage to all three regions. 22% of families are linked only to chromosome 4q and 17% are linked only to chromosome 17p. 13% of families are linked only to chromosome 11q. The greatest overlap is between families linked to 11q and 17p and between families linked to 4q and 11q. The results in FIG. 7 show that the identical regions of 11q having a maximal nonparametric score in both FIGS. 6B and 6C are not due to a highly overlapping set of families linked to 4q, 11q and 17p.

FIG. 8A includes a plot showing the results of a case/control association analysis. One individual was selected per family based on having a susceptibility haplotype on 4q within LPHN3 (forty-eight cases and forty controls; circles) or irrespective of haplotype within LPHN3 (squares). Filled rectangles represent haplotypes made of the most significant markers. Results are plotted as the −log(P value). Dashed line indicates significance as established by the Bonferroni correction (P_{corrected}=0.01/139=0.000072). Haplotype, rs677642-rs877137 was identified as very significant in this analysis (P<0.000005; GG; 57% allele frequency cases; 23% allele frequency controls). Under the plot is a diagram of the region encompassed by the haplotype as depicted by the University of California, Santa Cruz Genome Bioinformatics Group's UCSC genome browser.

FIG. 8B is a plot showing the results of a random effects model meta-analysis for the four indicated population samples. The meta-analysis results (Summary) confirms that the transmission of susceptibility markers in LPHN3 and in chromosome 11q are significantly associated (P<0.00001, OR=2.46 (95%, 1.68-3.70)).

FIGS. 9A, 9B, 9C, and 9D are plots showing the fittings of the indicated parameters (y-axis) based on a two-loci model (Interaction) or two single main effect models (11q haplotype and rs6551665). FIGS. 9A and 9B show fittings for myoinositol/creatinine levels in the right posterior cingulated gyrus and left posterior cingulated gyrus, respectively. FIG. 9C shows fittings for choline/creatinine levels in the right medial cingulate gyrus. In FIGS. 9A, 9B, and 9C, the fittings significantly improved using the two-loci model as compared to a single, main-effects model (P_{permuted}<0.05). The parameter in FIG. 9D represents the improvement in symptoms after treatment with methylphenidate as indicated by results for question number 18 on the SWAN scale (hyperactive question; “butting into conversations”) in eighty-two individuals from the USA1 sample. The i_{ad} coefficient for an interaction between an additive effect from chromosome 4 and a dominant effect from chromosome 11 indicated the better fitting model (P=0.027). In FIG. 9D, the fitting was also significantly improved using the two-loci model as compared to the single locus effect P_{permuted}=0.0036).

FIGS. 10A, 10B, 10C, and 10D are profile plots derived using latent class cluster analysis applied to symptoms of ADHD and associated disorders as measured by the Vanderbilt Assessment Scale for Parents (VAS-P) questionnaire. For each domain, VAS-P symptoms profiles within indicated clusters are shown in a scale from 1 to 4 (1=Never, 2=Occasionally, 3=Often and 4=Very often). Demographic characteristics for each cluster are shown in a scale from 0 to 1, representing the proportion of individuals from the population with such characteristics. FIG. 10A shows inattention symptoms measured by questions 1-9 (Q1-Q9)—Q1: careless, inattentive; Q2: sustains attention poorly; Q3: appears to not listen; Q4: poor follow through; Q5: disorganized; Q6: avoids/dislikes sustained mental effort; Q7: loses needed objects; Q8: easily distracted; and Q9: often forgetful. FIG. 10B shows hyperactivity/impulsivity (H/I) symptoms measured by questions 10-18 (Q 10-Q18)—Q10: fidgets or squirms; Q11: cannot stay seated; Q12: restless; Q13: loud; noisy; Q14: always “on the go”; Q15: talks excessively; Q16: blurts out; Q17: impatient; and Q18: intrusive. FIG. 10C shows oppositional defiant disorder (ODD) symptoms measured by questions 19-26 (Q19-Q26)—Q19: loses temper; Q20: argues with adults; Q21: defies adults' rules; Q22: annoys others; Q23: shifts blames to others; Q24: touchy; Q25: angry/resentful; and Q26: vindictive. FIG. 10D shows anxiety and depression (A/D) symptoms measured by questions 41-47 (Q41-Q47)—Q41: fearful, worried; Q42: fear of making mistakes; Q43: feels useless; Q44: blames self; Q45; feels unloved; Q46: sad; and Q47: embarrassed.

FIGS. 11A, 11B, 11C, 11D, 11E, and 11F are genotypic frequency distributions (Punnet squares) for pairs of SNPs (markers) which contribute to interaction effects as assessed by the Cochran-Mantel-Haenszel test. Epistatic effects are depicted by changes in darkness that represent significant differences in genotypic distribution among severe (cases) vs. not severe (controls) individuals. Lighter squares indicate a higher percentage of severe cases and darker squares indicate a higher percentage of not severe cases, as indicated by the adjacent scale bars (0.0 to 0.8). The genotype for one marker is held fixed while genotype on the other marker varies. FIG. 11A shows that markers rs1947275 in LPHN3 and rs17596017, in NCAM1, contribute to the severity of inattention symptoms (M²=33.163, FDR-corrected P-value<0.001). FIG. 11B shows that markers rs1947275 in LPHN3 and rs12799083 in DRD2 produce a significant interacting effect contributing to the severity of inattention symptoms (M²=28.456, FDR-corrected P-value<0.005). FIG. 11C shows that markers rs35106420 in LPHN3 and rs620291 in NCAM1 produce an interacting effect contributing to the severity of hyperactivity/impulsivity symptoms (M²=20.497, FDR-corrected P-value<0.05). FIG. 11D shows that markers rs995447 in LPHN3 and rsl 1214505 in NCAM1 interact to modify the severity of oppositional defiant disorder symptoms (M²=41.379, FDR-corrected P-value<0.0001). FIG. 11E shows that rs734644 in LPHN3 and rs620291 in NCAM1 produce an epistatic effect contributing to the severity of oppositional defiant disorder symptoms (M²=26.795, FDR-corrected P-value<0.01). FIG. 11F shows that markers rs1510920 in LPHN3 and rs4938006 localized near NCAM1 in an intragenic region of chromosome 11q interact to modify the severity of anxiety and depression symptoms (M²=41.379, FDR-corrected P-value<0.0001).

### DETAILED DESCRIPTION OF THE INVENTION

Single nucleotide variations in DNA sequences or single nucleotide polymorphisms (SNPs) are located in or close to genetic regions which are implicated in ADHD. SNPs are also stably transferred from generation to generation and thus can be used as biomarkers (“markers”) in population studies. The study of such markers is of great value for the medical and pharmaceutical communities as they can help evaluate the likelihood of developing ADHD, diagnose the severity of ADHD and its symptoms, and predict how an individual may respond to a given course of ADHD treatment. Thus, the discovery of markers associated with ADHD and associated disorders can be used to provide earlier and more accurate diagnoses and prognoses, provide opportunities for earlier intervention, and provide therapies that are tailored to the specific characteristics of disease in a patient.

The invention provides a method for determining whether a subject has an altered susceptibility to develop ADHD. The method includes analyzing a sample from the subject for the presence of one or more SNP alleles (markers) that are linked to the LPHN3 gene and one or more interacting SNP alleles (interacting markers) that are linked to a different gene on another chromosome. Interacting susceptibility markers can be located, for example, on chromosomes 11q, 5q, and 17p. The presence of (i) at least one susceptibility marker on chromosome 4q and (ii) at least one interacting susceptibility marker on chromosome 11q, 5q, or 17p indicates that the subject has an increased susceptibility to develop ADHD. As used herein, the term “increased susceptibility” means that the subject is more likely to develop ADHD than a subject without susceptibility markers.

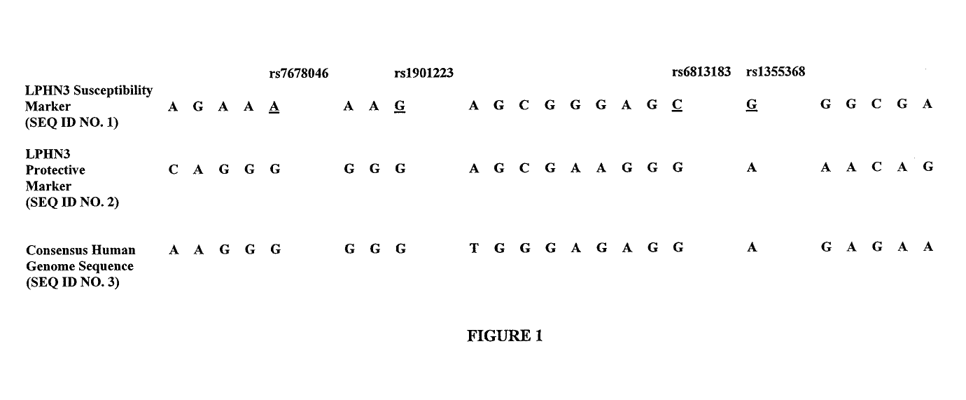

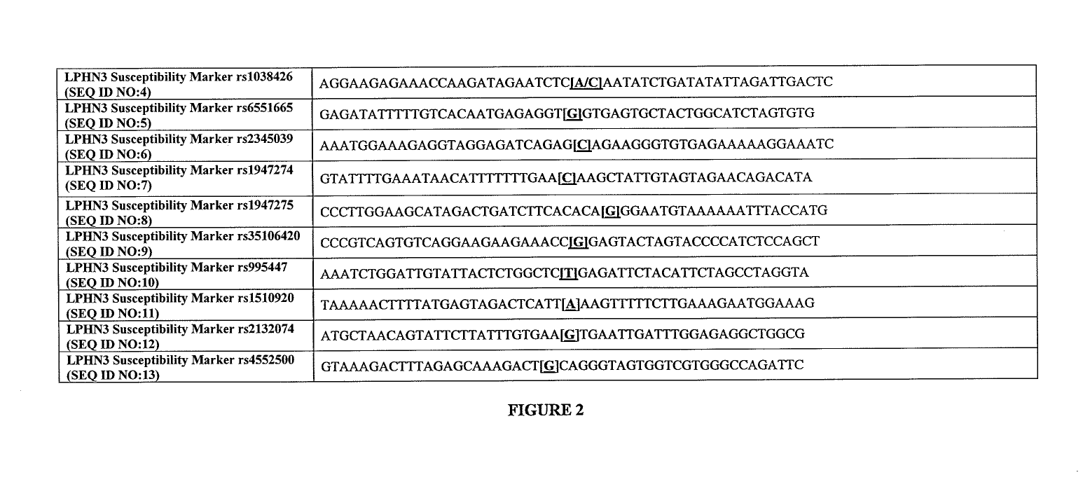

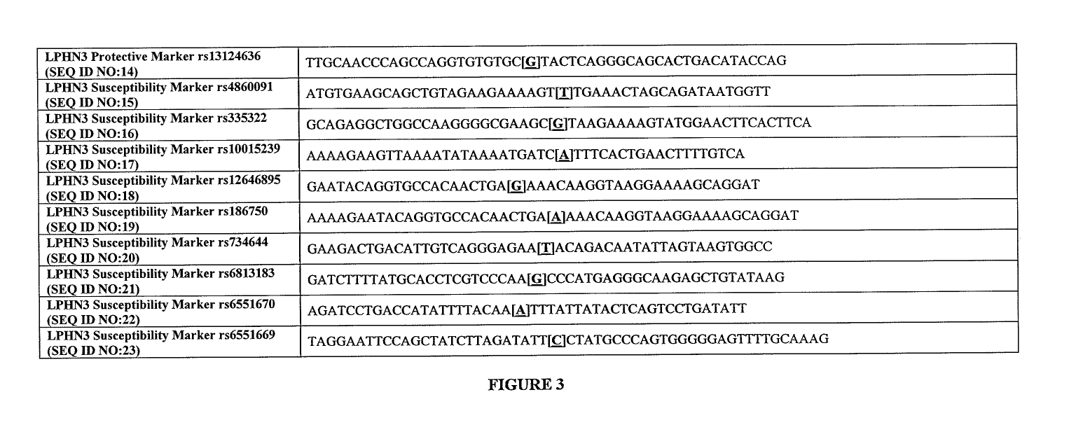

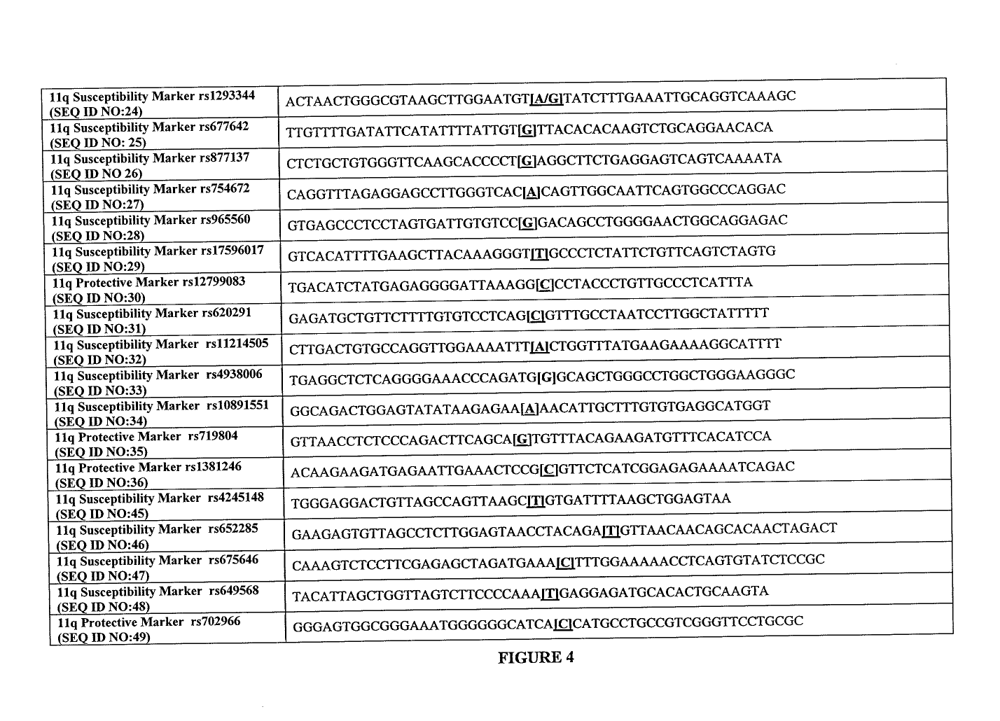

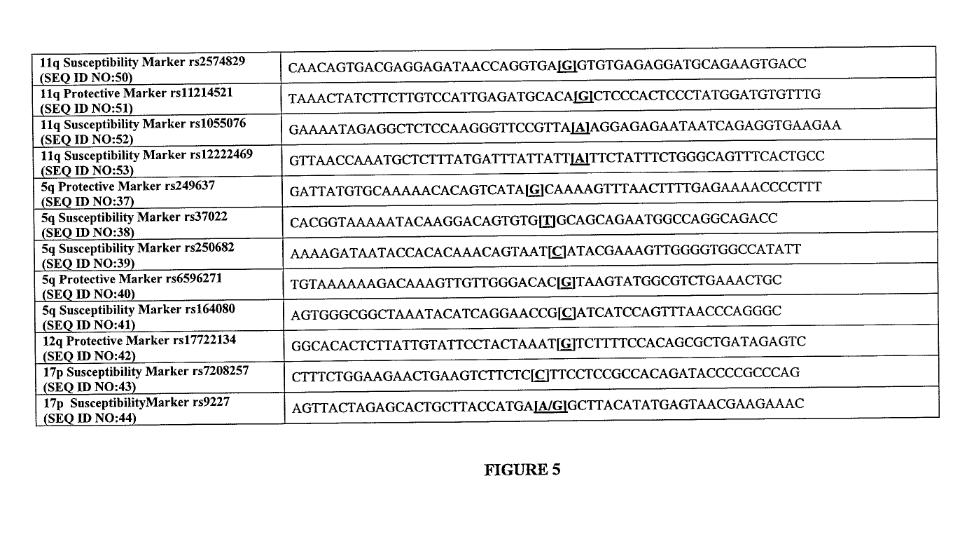

The marker linked to the LPHN3 gene can be located in the region spanning position 61,650,000-62,650,000 of chromosome 4q13.2 and the interacting marker can be located, for example, in the region spanning position 112,250,000-112,900,000 of chromosome 11q. Markers linked to LPHN3 include, for example, the SNP alleles identified as susceptibility or protective markers in FIGS. 1, 2, and 3, and in Tables 1 and 3. Interacting markers on chromosome 11q include markers linked to neural cell adhesion molecule 1 (NCAM1), the tetratricopeptide repeat domain 12 (TTC12), the ankyrin repeat and kinase domain containing 1 (ANKK1), and the 5′ UTR of the dopamine receptor D2 (DRD2). Interacting markers on chromosome 11 include, for example, the SNP alleles identified as susceptibility or protective markers in FIGS. 4 and 5, and in Tables 1 and 3. As demonstrated in the Examples below, susceptibility markers within LPHN3 (e.g., rs6551665) and interacting susceptibility markers in the indicated region of chromosome 11q are involved in ADHD susceptibility and substantially increase the risk for having ADHD by approximately 250% as compared to LPHN3 common variant markers.

To determine whether a subject has an increased susceptibility to develop ADHD, a sample from the subject is analyzed for (i) at least one ADHD susceptibility marker linked to the LPHN3 gene and (ii) at least one interacting ADHD susceptibility marker located on a second, different chromosome. The methods can include analyzing to determine whether the sample includes (i) more than one LPHN3 marker and/or (ii) more than one interacting ADHD susceptibility markers on another chromosome. Examples of susceptibility markers that can be used in the method of the invention include each of (i) the LPHN3 gene-linked SNP alleles in SEQ ID NOs: 1, 4-13, and 15-23 and (ii) the chromosome 11q SNP alleles in SEQ ID NOs: 24-34, 45-48, 50, 52, and 53; the chromosome 5q SNP alleles in SEQ ID NOs: 38, 39, and 41; and the chromosome 17p SNP alleles in SEQ ID NOs: 43 and 44.

In a method of determining whether a subject has an altered susceptibility to develop ADHD, the presence of one or more of the protective markers identified in FIGS. 1-5, and Tables 1 and 3, indicates that the subject is less susceptible to develop ADHD. The susceptibility and protective haplotypes in FIG. 1 are known in the art. Protective markers include, for example, protective markers on chromosomes 4q13.2 (SEQ ID NO:14), 11q (SEQ ID NOs: 35-36, 49, and 51), 5q (SEQ ID NOs: 37 and 70), and 12q (SEQ ID NO:42).

Having determined whether a subject has an increased susceptibility for development ADHD as disclosed herein, the invention further provides a method of monitoring and/or treatment. If the subject has an increased susceptibility to develop ADHD, the method can include increased or additional monitoring of the subject for the development of ADHD or worsening of ADHD symptoms. Alternatively or additionally, if the subject has an increased susceptibility to develop ADHD, the method can include providing an appropriate environment (e.g., a highly structured environment) and educating parents and/or caregivers regarding how to cope with and/or monitor the subject in view of the increased susceptibility to develop ADHD. Monitoring can beneficially provide for earlier intervention and treatment of the subject. Examples of increased or additional monitoring can include evaluating the subject with psychological or neurological diagnostic criteria for ADHD to identify symptoms of ADHD or related disorders. If the subject is determined to have an increased susceptibility for development ADHD as described herein, the method can further include providing the subject with treatment for ADHD. Treatment for ADHD can include pharmacological therapy, such as stimulants and antidepressants. Stimulants that can be used in the methods of the invention include, for example, clonidine, risperidone, modafinil. Antidepressants that can be used in the methods of the invention include, for example, bupropion and tricyclic antidepressants such as, e.g., desipramine, imipramine, and nortriptyline. Treatment for ADHD can also include behavioral or cognitive therapy techniques. Behavioral, cognitive, and pharmacological regimens can each be provided alone or, alternatively, two or more such regimens can be provided together as a combination therapy.

If the subject does not have an increased susceptibility for developing ADHD, the method can include providing treatment for a condition other than ADHD or, alternatively, providing no treatment as appropriate.

Additionally, the invention provides a method of determining whether a subject will benefit from treatment with a stimulant. An embodiment of the method includes analyzing a sample from the subject for at least one copy of an LPHN3 linked susceptibility marker (e.g., rs6551665 in SEQ ID NO:5) and two copies of an interacting susceptibility haplotype on 11q (e.g., rs677642 in SEQ ID NO:25 and rs877137 in SEQ ID NO:26 or rs754672 in SEQ ID NO:27 and rs965560 in SEQ ID NO:28). If the markers are present in the sample, the subject is considered a candidate for and likely to benefit from stimulant medication. The method can further include treating the subject with a stimulant. Alternatively, if sample from the subject does not include the LPHN3 linked susceptibility marker and has fewer than two copies of the susceptibility haplotype on 11q, the subject can be considered likely to have a poor response to stimulant treatment, and the method can further include treating the subject with therapy other than stimulant medication.

In another embodiment, the invention provides a method of determining or predicting the severity of symptoms of ADHD and associated disorders in a subject. The method includes analyzing a sample from the subject for the presence of one or more SNP alleles or markers that indicates the severity of one or more symptoms of ADHD or an associated disorder in the subject. Markers that indicate the severity of symptoms of ADHD and associated disorders in a subject include those shown in Table 3 below, e.g., markers linked to (i) the LPHN3 gene located in the region spanning positions 61,650,000-62,650,000 on chromosome 4q, (ii) the region spanning positions 112,250,000-112,900,000 of chromosome 11q, (iii) the NDFIP1 gene, SLC25A48 gene, or the GNPDA1 gene on chromosome 5q, (iv) the SLC6A3 gene on chromosome 5p, (v) the TPH2 gene on chromosome 12q, and (vi) the ARRB2 gene on chromosome 17.

The presence of the one or more ADHD susceptibility markers in the subject indicates that the subject has or is predicted to have a severe form of one or more symptoms of ADHD or an associated disorder. For example, according to the method of the invention, a subject is determined or predicted to have severe inattention symptoms when the sample from the subject is analyzed and found to have one or more susceptibility markers linked to one or more of the following genes: LPHN3 on chromosome 4q (e.g., rs1947275 in SEQ ID NO:8), NDFIP1 gene on chromosome 5q (e.g., rs249637 in SEQ ID NO:5), DRD2 on chromosome 11q (e.g., rs10891551 in SEQ ID NO:34), or the intergenic region between NCAM1 and TTC12 on chromosome 11q (e.g., rs719804 in SEQ ID NO:35). Additional susceptibility markers that are indicative of severe inattention symptoms include, for example, SNP alleles of rs652285 (in SEQ ID NO:46), rs675646 (in SEQ ID NO:47), and rs649568 (in SEQ ID NO:48), which are linked to NCAM1 on chromosome 11. See Table 3 below.

A subject is determined or predicted to be without severe inattention symptoms when the sample from the subject is analyzed and found to have one or more protective markers linked to the following genes: NDFIP1 on chromosome 5q (e.g., rs249637 in SEQ ID NO:37), TTC12 on chromosome 11q (e.g., rs719804 in SEQ ID NO:35), and PHRF1 on chromosome 11q (rs702966 in SEQ ID NO:49).

In another example, a subject is determined or predicted to have severe hyperactivity/impulsivity (H/I) symptoms when the sample from the subject is analyzed and found to have one or more susceptibility markers linked to one or more of the following genes: SLC6A3 on chromosome 5p (e.g., rs37022 in SEQ ID NO:38 or rs250682 in SEQ ID NO:39) or NCAM1 on chromosome 11q (e.g., rs652285 in SEQ ID NO:46, rs675646 in SEQ ID NO:47, or rs2574829 in SEQ ID NO:50). See Table 3 below.

In still another example, a subject is determined or predicted to have severe oppositional defiant disorder (ODD) symptoms when the sample from the subject is analyzed and found to have one or more susceptibility markers linked to one or more of the following genes: LPHN3 (e.g., rs995447 in SEQ ID NO:10, rs2132074 in SEQ ID NO:12, rs4552500 in SEQ ID NO:13, rs734644 in SEQ ID NO:20, 6813183 in SEQ ID NO:21, rs6551670 in SEQ ID NO:22, or rs6551669 in SEQ ID NO:23), NCAM1 on chromosome 11q (e.g., rs11214505 in SEQ ID NO:32), or TTC12 on chromosome 11q (e.g., rs1055076 in SEQ ID NO:52). See Table 3 below.

A subject is determined or predicted to be without severe ODD symptoms when a sample from the subject is analyzed and found to have one or more protective markers such as, for example, a protective SNP allele in the LPHN3 gene (e.g., rs 1312436 in SEQ ID NO:14) or on chromosome 11q (e.g., rs11214521 in SEQ ID NO:51 and rs11214505 in SEQ ID NO:32). See Table 3 below.

In a further example according to the method of the invention, a subject is determined or predicted to have severe conduct disorder (CD) symptoms when the sample from the subject is analyzed and found to have one or more susceptibility markers linked to one or more of the following genes: LPHN3 (e.g., rs4860091 in SEQ ID NO:15, rs335322 in SEQ ID NO:16, rs10015239 in SEQ ID NO:17), the intergenic region between NCAM1 and TTC12 on chromosome 11q (e.g., rs4938006 in SEQ ID NO:33), and the DRD2 gene on chromosome 11q (e.g., rs4245148 in SEQ ID NO:45). See Table 3 below.

A subject is determined or predicted to be without CD symptoms when a sample from the subject is analyzed and found to have one or more protective markers such as, for example, protective SNP allele in DRD2 on chromosome 11q (e.g., rs12799083 in SEQ ID NO:30).

In yet another example of the method of the invention, a subject is determined or predicted to have severe anxiety and depression (A/D) symptoms when the sample from the subject is analyzed and found to have one or more susceptibility markers linked to one or more of the following genes: LPHN3 (e.g., rs1510920 in SEQ ID NO:11, rs35106420 in SEQ ID NO:9, rs12646895 in SEQ ID NO:18, or rs186750 in SEQ ID NO:19), GNPDA1 on chromosome 5 (rs164080 in SEQ ID NO:41), or NCAM1 on chromosome 11q (e.g., rs652285 in SEQ ID NO:46, rs675646 in SEQ ID NO:47, rs649568 in SEQ ID NO:48, or rs12222469 in SEQ ID NO:53). See Table 3.

A subject is determined or predicted to be without severe A/D symptoms when a sample from the subject is analyzed and found to have one or more protective markers such as, for example, a protective SNP allele in the SLC25A48 gene (e.g., rs6596271 in SEQ ID NO:40) or in the intergenic region between NCAM1 and TTC12 on chromosome 11q (e.g., rs4938006 in SEQ ID NO:33). SNP rs4938006 is both a susceptibility marker for severe CD symptoms and a protective marker with respect to severe A/D symptoms.

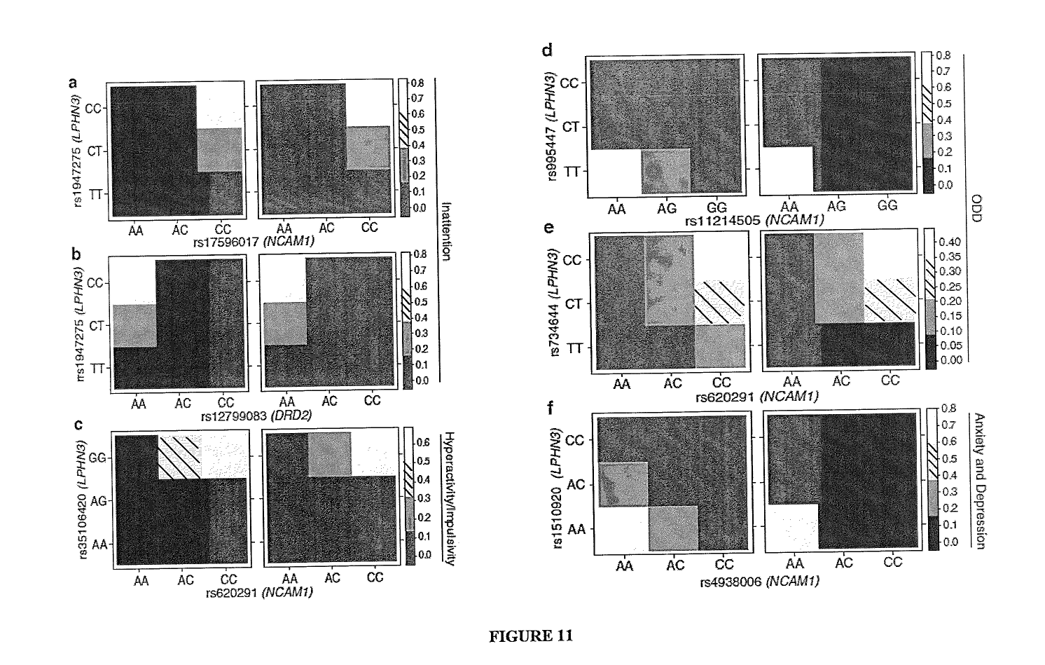

Moreover, combinations of the foregoing interacting markers have been found to be indicative of the severity of symptoms of ADHD and associated disorders. For example, two-locus, cooperative interactions between genetic markers located on 4q13.2 (i.e., LPHN3) and chromosome 11q which are indicative of particular ADHD symptoms are shown in FIG. 11 and discussed in Example 6 below.

The disclosed method of determining or predicting the severity of symptoms was derived using severity determinations according to two methods. As described more fully in the Examples below, symptom severity was determined using an overall VAS-P score and was also determined by quantifying the magnitude of the following symptoms of ADHD and ADHD-associated disorders: Inattention, Hyperactivity/impulsivity, CD, ODD and A/D symptoms using latent class cluster analysis (LCCA). In this way, the severity determinations disclosed herein included all clinical information from the individuals regardless of their categorically diagnosed status. Accordingly, the method of the invention is particular useful in a clinical context, where severity of symptoms is a major predictor of functional outcome for a patient.

After determining or predicting the severity of one or more symptoms of symptoms of ADHD or an associated disorder for a subject as disclosed herein, the invention further provides a method of monitoring and/or treatment. If the subject is determined or predicted to have one or more severe symptoms of ADHD or an associated disorder, the method can include monitoring the subject for the development or exacerbation of one or more symptoms of ADHD or an associated disorder. Increased monitoring can beneficially provide an earlier intervention and treatment of the subject. If the subject is determined or predicted to have one or more severe symptoms of ADHD or an associated disorder, the method can further include providing the subject with treatment that is tailored for the one or more severe symptoms. Treatment tailored for one or more severe symptoms of ADHD or an associated disorder can include pharmacological therapy, such as stimulants and antidepressants. Stimulants that can be used include, for example, clonidine, risperidone, modafinil. Antidepressants that can be used include, for example, bupropion and tricyclic antidepressants such as, e.g., desipramine, imipramine, and nortriptyline. For example, a subject that is determined or predicted to have severe anxiety/depression symptoms can be treated with antidepressants.

Treatment can also include behavioral or cognitive therapy techniques that are specifically tailored or targeted for the one or more severe symptoms of ADHD or an associated disorder. For example, a subject that is determined or predicted to have one or more severe hyperactivity/impulsivity, CD, ODD, or A/D symptoms can benefit from behavioral or cognitive therapy techniques specifically targeted to the one or more severe symptoms. Tailored behavioral, cognitive, and pharmacological treatment regimens can each be provided alone or, alternatively, two or more such regimens can be provided together as a combination therapy.

If the subject is determined or predicted not to have one or more severe symptoms of ADHD or an associated disorder, the method can include tailoring therapy to exclude unnecessary therapy for symptoms that the subject does not and is not predicted to have.

For purposes of the present invention, the subject can be any mammal and can include, without limitation, mammals ordinarily treated in the course of veterinary care and mammalian models of ADHD such as rats, mice, and primates. An especially preferred subject is human. The subject can be a child, e.g., a human under the age of 18 years, or a child that is under the age of 17 years, 16 years, 15 years, 14 years, 13 years, 12 years, 11 years, 10 years, 9 years, 8 years, 7, years, 6 years, 5 years, 4 years, 3 years, 2 years, or 1 year old.

The sample may be may be any fluid or tissue obtained from the subject that contains genetic material. Examples of suitable samples can include, but are not limited to, whole blood, blood plasma, blood serum, urine, saliva, cells (e.g., cells obtained from blood, such as epithelial cells), and tissue. Preferred samples include brain tissue and blood.

The sample may be analyzed by any suitable method for determining the relevant SNP marker sequences. For instance, one way of analyzing the sample is to amplify the genetic material contained in the sample using techniques known in the art, such as polymerase chain reaction (PCR), and subsequently sequencing the sample to determine if the subject carries the one or more ADHD susceptibility markers or protective markers. Methods of gene sequencing are well known to those having ordinary skill in the art. Specific SNP alleles can be analyzed (in native genomic DNA or in PCR amplified material) using restriction endonucelases that preferentially digest DNA having a recognition sequence which includes the relevant SNP allele in techniques that are based on restriction fragment length polymorphisms (RFLPS). Other methods that may be used to detect one or more SNPs include techniques based on fluorescence in situ hybridization (FISH) and matrix-assisted laser desorption/ionization time-of-flight mass spectrometry (MALDI-TOF-MS). Alternatively, SNP marker sequences may be detected and analyzed in the sample, e.g., using nucleic acid probes and arrays. Nucleic acid arrays are described, for example, in U.S. Pat. Nos. 5,981,956, 5,922,591, and 5,994,068. Arrays using DNA chips and tiled nucleic acid arrays, as well as additional methods of SNP detection, are described in U.S. Patent Publication No. 2003/0211500A1.

Since SNPs constitute sites of variation flanked by regions of relatively invariant sequence, the analysis of a susceptibility or protective marker disclosed herein requires no more than the determination/identification of the single nucleotide present at the site of variation. Therefore, it is unnecessary to determine a complete gene sequence and it is also unnecessary to determine all of the nucleotides referenced in any of the sequence identifiers (i.e., SEQ ID NOs: 1-53) disclosed herein. Depending on techniques used, the presence or absence of a SNP may be determined by analyzing a sequence of only, for example, four, five, six, seven, or eight contiguous nucleotides that includes the single nucleotide polymorphism in a sequence disclosed herein. For example, certain restriction enzymes have recognition sequences with as few as four, five, six, seven, or eight nucleotides. Moreover, in view of the potential variability of genomic sequence flanking a SNP, the method of the invention can include analyzing sequences with a minimum identity of 90%, 91%, 92%, 93%, 94%, 95%, 96%, 97%, 98%, or 99% to the SNP-flanking sequence disclosed herein. The analyzed SNP flanking sequence can include, for example, two or more, three or more, four or more, five or more, six or more, seven or more, eight or more, nine or more, ten or more, eleven or more, or twelve or more nucleotides flanking each side of the SNP within any one of SEQ ID NOs: 1-53.

Furthermore, whenever the present disclosure refers to the analysis of a sample for a susceptibility or protective marker within a sequence identifier (i.e., any one of SEQ ID NOs: 1-53), it should be apparent that the method can also include analyzing the sample for the susceptibility or protective marker within a sequence that is complementary to the disclosed sequence identifier. This is because SNPs are necessarily present on both chromosomal strands and, therefore, each of the susceptibility and protective markers disclosed herein can be identified on the plus or on the minus chromosomal strand.

The following examples further illustrate the invention but, of course, should not be construed as in any way limiting its scope.

### Example 1

This example demonstrates that ADHD susceptibility markers in LPHN3 interact with markers in chromosome regions 11q and 17p.

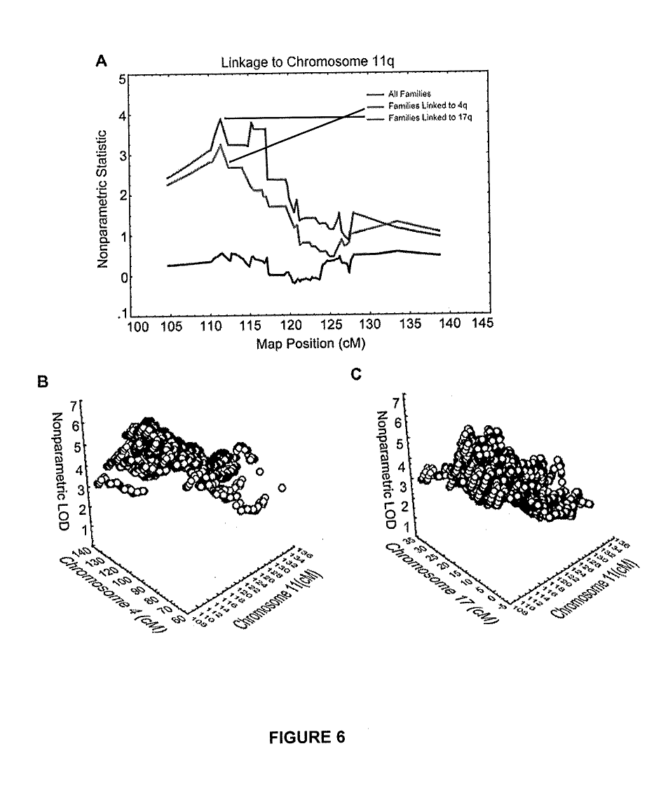

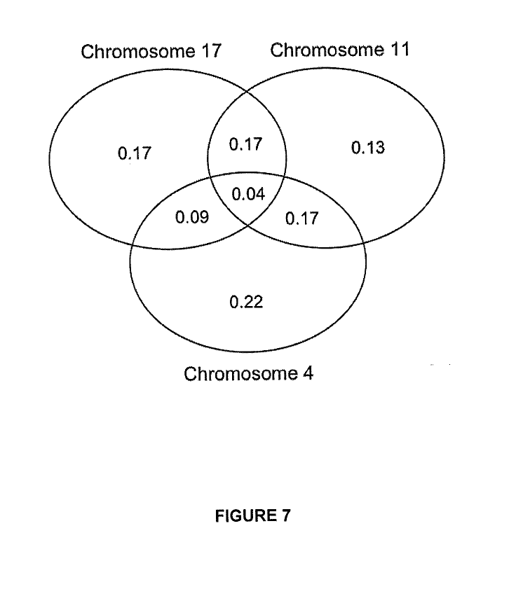

To screen for interacting regions on chromosomes 5q, 8q, 11q, and 17p, correlation subset analysis was performed. Cox et al., Nat. Genet., 21 (2): 213-5 (1999). The analysis involved one hundred and thirty four nuclear families from the Paisa genetic isolate and single nucleotide polymorphisms (SNPs) spanning each minimal critical regions (MCR) with a resolution of 200 kb. Mapping resolution and SNPs selection criteria are published in Arcos-Burgos et al., Mol. Psychiatry, 15(11): 1053-66 (2010). Non-parametric linkage score (NPL) was calculated using GENEHUNTER (Kruglyak et al., Am. J. Hum. Genet. 58(6): 1347-63 (1996)) with the weight function weight₁₋₀. Families that demonstrated nominal non-parametric linkage (NPL>1.00) to a region were included in the analysis (i.e., were coded as 1 for weight₁₋₀) and families that did not were excluded (i.e., were coded as 0 for weight₁₋₀). Cox et al., Nat. Genet., 21(2): 213-5 (1999). Heterogeneity was measured using the weight function weight₁₋₀ (defined analogously), where only those families that did not demonstrate linkage to a region were included in the analysis. Of the entire set of possible pair-wise comparisons, only two showed an increase in the NPL statistic greater than two units when compared to linkage analyses using all families. Families linked to 4q showed an increase in the nonparametric linkage statistic from 0.55 to 3.24 in an overlapping region on 11q (4q-11q interaction) and families linked to 17p showed an increase in the nonparametric linkage statistic from 0.55 to 3.88 on 11q (17p-11q interaction). These linkage relationships are shown in FIG. 6A.

The 4q-11q interaction of SNPs rs1038426 (4q) and rs1293344 (11q) produced a maximal nonparametric score of 6.08 (P<1×10⁻⁸) (see FIG. 6B) and the 17p-11q interaction of SNPs rs9227 (17p) and rs1293344 (11q) produced a maximal nonparametric score of 5.51 (P<1×10⁻⁶) (see FIG. 6C) when analyzed using GENEHUNTER-TWOLOCUS. Strauch et al., Am. J. Hum. Genet., 66(6): 1945-57 (2000), Dietter et al., Eur. J. Hum. Genet., 12(7): 542-50 (2004). Two locus parametric analyses revealed convergent evidence of interactions. The discovery sample proved to be a powerful one for revealing two-locus interactions when using power analyses to detect two interacting loci (considering ADHD as a binary trait) and also for detecting interacting loci underlying quantitative traits.

Throughout the scanned region there was found to be a remarkable absence of linkage disequilibrium (LD) gaps, suggesting that the genotyped markers used in this study covered any variation inside of the 4q-linked region. Scanning was done using a very stringent LD map, with a resolution of ˜68 kb, which is two times stronger that the resolution recommended for covering this genomic area. Arcos-Burgos et al., Mol. Psychiatry, 15(11): 1053-66 (2010). This map was used to identify a unique region of association and linkage of ˜240 Kb that was harbored inside of the LPHN3 gene. No other areas inside of the 4q-linked region showed a positive association. Additional analysis has shown that the LPHN3 common variant confers susceptibility to ADHD, affects brain metabolism, and predicts effectiveness of stimulant medication. Arcos-Burgos et al., Mol. Psychiatry, 15(11): 1053-66 (2010). Three markers harbored in LPHN3 passed the test of heterogeneity and were significant after adjusting for multiple tests: rs6551665 (G, allele) in SEQ ID NO:5 (OR=1.23, 95% CI 1.09-1.37, P=3.46×10⁻⁴), rs1947274 in SEQ ID NO:7 (OR=1.23, 95% CI=1.09-1.38, P=5.41×10⁻⁴), rs2345039 in SEQ ID NO:6 (OR=1.21, 95% CI=1.08-1.35, P=8.97×10⁻⁴). The marker rs6551665 was genotyped in all the samples and the LD r² (square of the correlation coefficient) with each of these other two markers was greater than 90%. Therefore, this marker was used as a proxy to evaluate the association of ADHD and LPHN3, instead of the markers reported by Arcos-Burgos et al., Mol. Psychiatry, 15(11): 1053-66 (2010).

The marker on 11q by itself did not exhibit main effects. Its association with ADHD is demonstrated when conditioning on the susceptibility variant within LPHN3. Thus the marker on 11q appears to act as a modifier of LPHN3 susceptibility.

An association analysis was performed using SNPs on 11q and 17p, conditioned on being a carrier of the susceptibility allele at rs6551665 (G variant). Either one case or control with at least one copy of the LPHN3 susceptibility allele rs6551665 (G variant) was selected per family. Due to the rarity of individuals who are homozygous for the rs6551665 G allele, they were pooled with G-heterozygotes. Using individuals who are carriers of the G variant of susceptibility at rs6551665, the association signals at 11q and 17p were narrowed.

The foregoing results show that ADHD susceptibility markers in LPHN3 and interacting markers in each of chromosome regions 11q and 17p are indicative of an increased susceptibility for ADHD.

### Example 2

This example shows that the cooperative interaction between ADHD susceptibility markers in LPHN3 and interacting susceptibility markers on chromosome 11q is maintained in multiple populations.

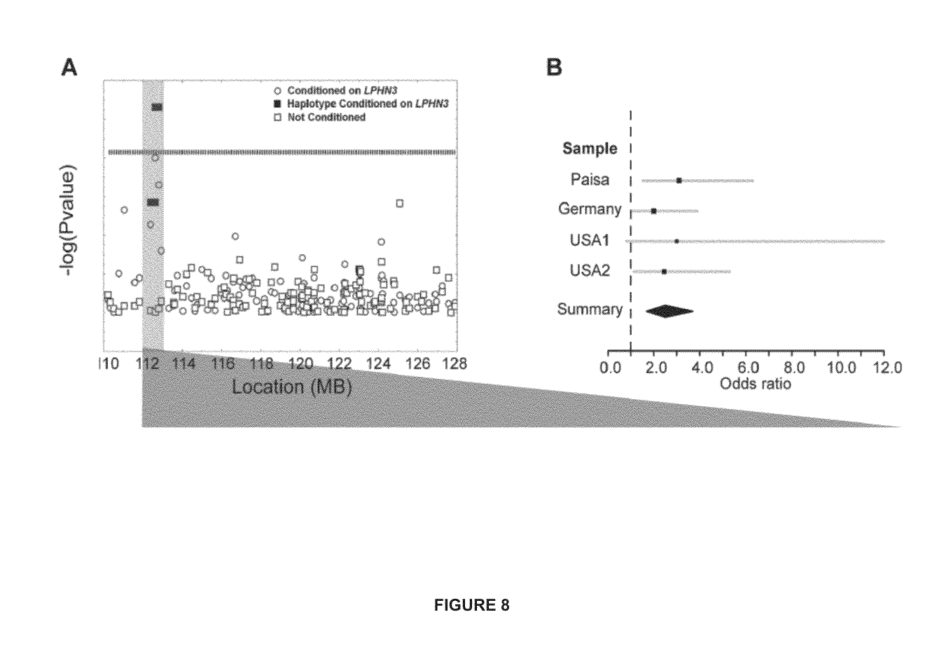

Single marker and haplotype case-control association analysis was employed to isolate a single strongly associated haplotype on chromosome 11q that included rs677642 (G variant) in SEQ ID NO:25 to rs877137 (G variant) in SEQ ID NO:26 (OR=4.47 CI 2.30-8.69, P<0.000005, P_{corrected}<0.005). See FIG. 8A and Table 1A. The haplotype spanned 166 kbps, from intron 7 of the gene coding neural cell adhesion molecule 1 (NCAM1), encompassed the tetratricopeptide repeat domain 12 (TTC12) and the ankyrin repeat and kinase domain containing 1 (ANKK1), and was adjacent to the 5′ UTR of the dopamine receptor D2 (DRD2) as shown in FIG. 8A. Transmission disequilibrium test (TDT) analysis of the entire set of multigenerational, nuclear, and trio Paisa families was performed to avoid the potential effects of genetic stratification, which can be a source of Type I error. The odds ratio (OR) for the transmission of the susceptibility variants on 4q and 11q was 3.14 (95% IC-1.49-6.62; P<0.0027) compared to transmission of neither variant. Transmission frequencies, OR, and P-values in each of the populations samples are shown in Table 1B. Both the association and TDT analyses demonstrated that the significance of the association on chromosome 11q is lost when not conditioning on the presence of the susceptibility variant within LPHN3.

TABLE 1A

| | 11q | Cases | Controls | | |
|---|---|---|---|---|---|
| LPHN3* | Haplotype** | (Freq) | (Freq) | OR (95% CI) | P-value |
| G | GG | 55 (57%) | 18 (23%) | 4.47 (2.30, 8.69) | P < 0.000005, |
| G | Else | 41 (43%) | 60 (77%) | | P_{corrected} < 0.005 |

TABLE 1B

| | | | | | Not | | | |
|---|---|---|---|---|---|---|---|---|
| | | 11q- | Transmitted | | Transmitted | | OR | |
| Sample | LPHN3* | Haplotype*** | (Freq) | | (Freq) | | (95% CI) | P-value |
| Paisa | G | GG | 31 | (10.2%) | 10 | (3.3%) | 3.14 | 0.0027 |
| | | | | | | | (1.49, 6.62) | |
| | G | Else | 39 | (12.9%) | 27 | (8.9%) | 1.46 | 0.1661 |
| | | | | | | | (0.85, 2.51) | |
| | Else | GG | 78 | (25.7%) | 108 | (35.8%) | 0.73 | 0.0943 |
| | | | | | | | (0.51, 1.06) | |
| | Else | Else | 155 | (51.2%) | 157 | (52%) | Reference | Reference |
| German | G | GG | 26 | (7.9%) | 13 | (3.9%) | 1.91 | 0.0706 |
| | | | | | | | (0.95, 3.84) | |
| | G | Else | 58 | (17.6%) | 50 | (15.2%) | 1.11 | 0.6505 |
| | | | | | | | (0.72, 1.71) | |
| | Else | GG | 75 | (22.7%) | 104 | (31.5%) | 0.69 | 0.0449 |
| | | | | | | | (0.48, 0.99) | |
| | Else | Else | 171 | (51.8%) | 163 | (49.4%) | Reference | Reference |
| US1 | G | GG | 9 | (7.9%) | 3 | (2.6%) | 3.1 | 0.1014 |
| | | | | | | | (0.8, 12) | |
| | G | Else | 19 | (16.7%) | 16 | (13.9%) | 1.23 | 0.5955 |
| | | | | | | | (0.58, 2.59) | |
| | Else | GG | 23 | (20.2%) | 31 | (27%) | 0.77 | 0.4139 |
| | | | | | | | (0.4, 1.45) | |
| | Else | Else | 63 | (55.3%) | 65 | (56.5%) | Reference | Reference |
| US2 | G | AG | 22 | (5.7%) | 9 | (2.4%) | 2.28 | 0.0432 |
| | | | | | | | (1.03, 5.08) | |
| | G | Else | 108 | (28.4%) | 123 | (32.4%) | 0.82 | 0.2279 |
| | | | | | | | (0.59, 1.13) | |
| | Else | AG | 40 | (10.5%) | 52 | (13.7%) | 0.72 | 0.1306 |
| | | | | | | | (0.46, 1.13) | |
| | Else | Else | 210 | (55.3%) | 196 | (51.6%) | Reference | Reference |

TABLE 1C

| Meta-analysis | OR (95% CI) | P-value |
|---|---|---|
| Interaction | 2.46 (1.68, 3.70) | <0.00001 |
| LPHN3 only* | 1.04 (0.86, 1.25) | 0.7111 |
| Haplotype only*** | 0.73 (0.61, 0.87) | <0.001 |

*Defined by the marker rs6551665 in chromosome 4
**Defined by the markers rs677642 and rs877137 in chromosome 11
***Defined by the markers rs677642 and rs877137 in chromosome 11 except in the US2 sample, for which it is defined by the markers rs754672 and rs965560

TDT results after combining 4q-11q variants produced a decrease of the original sample size, consistent with a previous report. Arcos-Burgos et al., Mol. Psychiatry, 15(11): 1053-66 (2010). The decrease of the sample size is a consequence of the ad hoc strategy of conditioning upon the fact of being a carrier of the G variant of the susceptibility marker at rs6551665. Furthermore, because of genotype limitations, the Norwegian and Spaniard samples were not genotyped for markers in 11q. Given that TDT was selected to evaluate interaction effects, information contained in the original family structure sample was lost.

To confirm the cooperative interaction in other population samples, TDT analyses were performed on three additional samples: one from Germany and two primarily European-American samples consisting of ninety five trios collected at the NHGRI, Bethesda, Md., USA (US1) and two hundred and forty trios from a sample collected at Children's Hospital of Philadelphia, Philadelphia, Pa., USA (US2) (Table 1B). All three samples were used to confirm the LPHN3 association to ADHD. Arcos-Burgos et al., Mol. Psychiatry, 15(11): 1053-66 (2010). The US2 sample was not genotyped at identical SNPs on 11q and, therefore, a two tag-SNPs that fully describe the variation (r²=1, rs754672, rs965560) was tested for the European “CEU sample” assembled by the International Haplotype Mapping Project or HapMap Consortium, 2005. These results show a similar pattern of interaction and confirm that the haplotype on 11q interacts cooperatively with the LPHN3 susceptibility variant to increase the risk to ADHD.

A meta-analysis of the TDT results from the four samples (Paisa, Germany, US1, and US2) was performed using a random effects model. The meta-analysis also found a significant association in the transmission of both susceptibility variants on chromosome 4q and 11q (OR=2.46, 95% CI-1.63-3.70, P<0.00001). See FIG. 813 and Table 1C above.

The foregoing results confirm that the cooperative interaction between ADHD susceptibility markers in LPHN3 and interacting susceptibility markers on chromosome 11q is indicative of ADHD susceptibility in multiple populations.

### Example 3

This example demonstrates that the LPHN3-11q cooperative interaction modulates brain metabolism.

Proton magnetic resonance spectroscopy (¹H-MRS) was performed on eighteen individuals from the Paisa genetic isolate to evaluate four metabolites N-acetylaspartate, myoinositol, choline and glutamine (for all taken as the ratio to creatine), in several brain regions making up part of the frontal striatal-cerebellar circuit. A full two-locus interaction model (Cordell et al., Hum. Mol. Genet. 11(20): 2463-8 (2002) Cordell et al., Genetics, 158(1): 357-67 (2001)) was fit to the data using linear regression, where y is the quantitative MRS metabolite phenotype, μ is the mean effect, A is the age at diagnosis, S is a code for gender (males=0, females=1), D describes disease status (unaffected=0, affected=1), xᵢ, i=1, 2, is a variable modeling an additive effect (−1 for homozygote for allele 1, 0 for a heterozygote and 1 for a homozygote for allele 2), zᵢ is a dummy variable for a dominant effect (−0.5 for homozygote for allele 1, 0.5 for a heterozygote and −0.5 for a homozygote for allele 2), aᵢ and dᵢ refer to additive and dominant coefficients estimated for the single locus effect, and iₐₐ, i_{ad}, i_{da} and i_{dd} represent epistatic coefficients in the following model:

y=μ+S+A+D+a₁x₁+d₁z₁+a₂x₂+d₂z₂+iₐₐx₁x₂+i_{ad}x₁z₂+i_{da}z₁x₂+i_{dd}z₁z₂

This model was compared to a nested model lacking interaction coefficients using a likelihood ratio test (LRT) that follows a χ²-distribution with four degrees of freedom. Three regions gave results that were significant after permutation (P_{permuted}<0.05): myoinositol in the right posterior cingulate gyrus, myoinositol in the left posterior cingulate gyrus, and choline in the right medial cingulate gyrus.

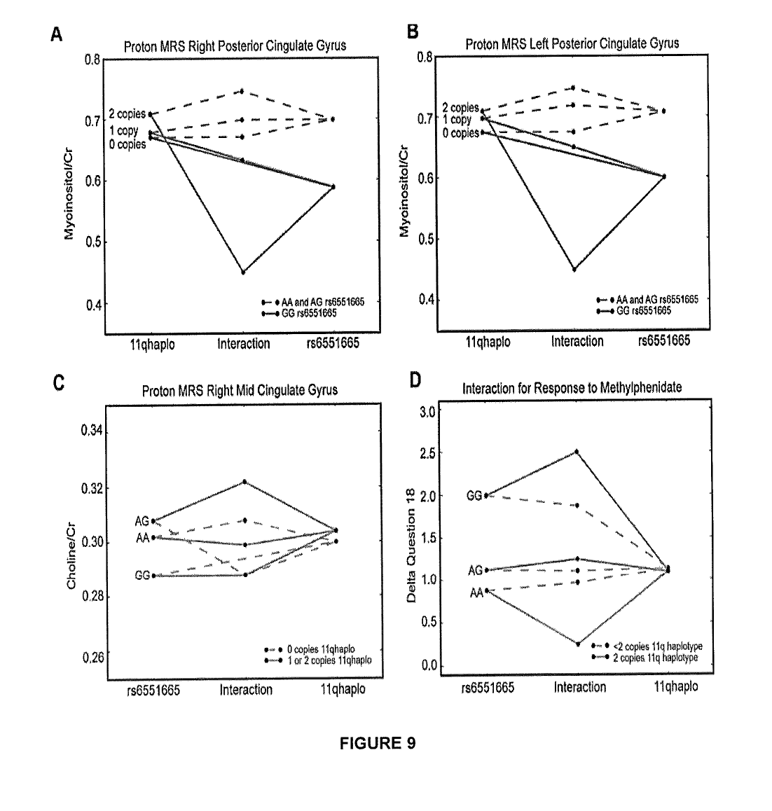

For myoinositol in the right and left posterior cingulated gyrus, each i_{ad} coefficient for an interaction between an additive effect from the haplotype on 11q and a dominant effect from rs6551665 contributed to the better fitting model (i_{ad} left cingulate gyrus P=0.00342; i_{ad} right cingulate gyrus P=0.00298). Therefore, the means were plotted for an additive effect from the 11q haplotype and a dominant effect from rs6551665 (where values from individuals that were AA or AG at rs6551665 were grouped). The results demonstrated that having two copies of the susceptibility haplotype on chromosome 11 and GG at rs6551665 (i.e., having two copies of rs6551665 in SEQ ID NO:5) correlates with a significant decrease in myoinositol in these two regions (FIGS. 9A and 9B).

For choline in the right medial cingulated gyrus, the i_{ad} coefficient for an interaction between an additive effect from rs6551665 and a dominant effect from the 11q haplotype contributed to the better fitting model (P=0.00968). Therefore, the results were plotted for an additive effect from rs6551665 and a dominant effect from the haplotype on 11q (where having one or two copies of the susceptibility haplotype were grouped). The results demonstrated that having AG at rs6551665 (i.e., being heterozygous for rs6551665) and at least one copy of the susceptibility haplotype on 11q is related to an increase in choline in the right medial cingulate and that having AG at rs6551665 and no copies of the susceptibility haplotype on 11q is related to a decreased level of choline in the right medial cingulate region (FIG. 9C).

This foregoing results show that the cooperative interaction of LPHN3-11q markers modulate metabolism of myoinositol and choline in the brain.

### Example 4

This example demonstrates pharmacogenetic effects of the LPHN3-11q cooperative interaction, including the consequences of susceptibility variants on treatment response to stimulant medication.

Eighty-two individuals with complete genotype and phenotype information were sampled from the original 240 trios in the US1 sample of Example 1. A comparative analyses regarding stimulant treatment was done as described in Arcos-Burgos et al., Mol. Psychiatry, 15(11): 1053-66 (2010). An analysis was performed to determine the relationship of SWAN scale (Strengths and Weaknesses of ADHD Symptoms and Normal Behavior scale) questions individually as well as inattentive and hyperactive combined dimensions (questions 1-9 indicate inattentive symptoms and questions 10-18 indicate hyperactive symptoms), prior to and after starting treatment with the stimulant methylphenidate. A significantly better fitting model was obtained for question 18 (hyperactive-impulsive dimension) when the i_{ad} coefficient for interaction between additive effects at LPHN3 and dominant effects at 11q are included (P_{corrected}−0.0036). The results demonstrated that having GG at rs6551665 and two copies of the susceptibility haplotype on 11q correlated with a significant improvement of symptoms after treatment with stimulant medication. Results also demonstrated that having AA at rs6551665 and fewer than two copies of the susceptibility haplotype on 11q correlated with a poor response to stimulant medication treatment. See FIG. 9D. Larger studies are expected to more precisely define the statistically significant effects demonstrated herein for the relationship between LPHN3-11q interaction on treatment response to stimulant medication.

The foregoing results show that the cooperative interaction among LPHN3-11q markers disclosed herein can be used to determine whether a patient is a candidate for and likely to benefit from stimulant medication or, alternatively, whether the patient is likely to respond poorly to stimulant medication.

### Example 5

This example identifies a number of genetic markers that are predictors of the severity of symptoms of ADHD and associated disorders, including markers linked to LPHN3 (4q), NDFIP1 (5q), DRD2 (11q), TTC12 (11q), TPH2 (12q), SLC6A3 (5p), GNPDA1 (5), and NCAM1 (11q).

Subjects

Three hundred and forty-nine nuclear families consisting of a total of 1,371 individuals were analyzed. Participants were from the United States, four to sixty-five years of age, and ascertained from ADHD probands. Diagnosis of ADHD in children was established using the DSM-IV criteria (American Psychiatric Association, Diagnostic and Statistical Manual of Mental Disorders 4^{th} Ed., rev. (2000)); in adults, the Conners Adult ADHD Rating Scale (CAARS) (Conners et al., The Canners Adult ADHD Rating Scale (CAARS), Multi-Health Systems Inc. (1998)) was used. All participants were evaluated using the Vanderbilt Assessment Scale for Parents (VAS-P) (Wolraich et al., J. Pediatr. Psychol., 28(8): 559-67 (2003)), which includes all eighteen DSM-IV criteria for ADHD (questions 1-9 for inattention; questions 10-18 for hyperactivity/impulsivity(H/I), questions 19-26 for oppositional defiant disorder (ODD), and questions 27-40 for conduct disorder (CD)) and seven items from the Pediatric Behavior Scale (Lindgren et al. in Advances in Behavioral Assessment of Children and Families, p. 57-90, Prinz R., Ed., JAI Press (1987). Participants were also screened for anxiety and depression (A/D) (questions 41-47 of the VAS-P). Each question of the VAS-P is scored based on the frequency of presentation of the behavior on a numerical scale (1=Never, 2=Occasionally, 3=Often and 4=Very often). Wolraich et al., J. Pediatr. Psychol., 28(8): 559-67 (2003). Complete information was available for 1,341 individuals. More detailed information regarding clinical assessment and specific demographics of this sample has been published. Acosta et al., J. Am. Acad. Child Adolesc. Psychiatry 47(7): 797-807 (2008).

Severity Determination:

Latent class cluster (LCCA) models containing one to ten different classes were fitted to the data using Latent GOLD 4.5 (Statistical Innovations, Belmont, Mass.). Latent GOLD uses both EM and Newton-Raphson algorithms to find the maximum likelihood for each model after estimating model parameters. Vermunt et al, Applied latent class analysis, Cambridge University Press (2002). In the LCCA models, separate analyses were performed for each of the five VAS-P domains (inattention, H/I, ODD, CD and A/D). The number of clusters was selected using a likelihood ratio test (LRT) evaluating whether increases in likelihood (L²) associated with increased latent classes justified their inclusion. Certainty of these clusters was assessed calculating P-values associated with L² values after running 500 parametric bootstrap replicates. Categorical variables of gender, ADHD diagnosis, and age (children: four to eleven years old; adolescents: twelve to seventeen years old; adults older than seventeen years old) were used as covariates for all models. Arcos-Burgos et al., Mol. Psychiatry; 15(11): 1053-66 (2010).

As implemented in Latent GOLD, individuals were assigned posterior membership probabilities for belonging to each cluster based on their symptom profiles. Individuals were additionally assigned to the cluster for which the posterior probability was highest. Based on this assignment, VAS-P profiles were obtained for each cluster. Using these profiles, a severity scale was derived as follows: consider the LCCA results for a particular VAS-P domain, e.g. inattention, and let K be the number of clusters found, Q be the number of questions in that domain, nᵢ be the number of individuals in that cluster (i=1, 2, . . . , K), and N_{P}ⁱ be the number of questions in cluster i with an average profile P. If N_{P>2}ⁱ>Q/2, all nᵢ individuals in cluster i were classified as being severe for this domain, and not severe otherwise. In other words, individuals with severe symptoms were those for whom the cluster they belong to, had at least Q/2+1 questions with a profile higher than 2, i.e., more than “Ocassionally.” Individuals with severe symptoms were treated as “cases” and non-severely affected individuals as “controls.” For the analyses, this derivation allowed more precise descriptions of each individual's characteristics (profiles), descriptions that would be difficult to define when relying solely on the categorical DSM-IV diagnosis.

Genotyping and Genetic Statistical Analysis:

DNA was prepared from peripheral-blood specimens and the Illumina (San Diego, Calif., USA) genotyping service was used for genotyping, as described in Arcos-Burgos et al., Mol. Psychiatry; 15(11): 1053-66 (2010). Out of the 1,341 individuals who completed the VAS-P questionnaire, blood samples were available for 1,181; genetic data was available for 813 individuals. A total of three hundred and sixty-nine markers homogenously distributed on genomic regions of chromosome 4 (one hundred and fifty-six markers), chromosome 5 (forty-eight markers), chromosome 10 (seven markers), chromosome 11 (one hundred and twenty nine markers), chromosome 12 (fifteen markers) and chromosome 17 (fourteen markers), as previously described in Arcos-Burgos et al., Mol. Psychiatry; 15(11): 1053-66 (2010), were selected for analysis. See Arcos-Burgos et al., Am. J. Hum. Genet.; 75(6): 998-1014 (2004).

Family-based association tests (FBATs) using a dominant model under the hypotheses of no linkage and no association were performed as implemented in the PBAT module of Golden Helix PBAT Software (Golden Helix, Inc., Bozeman, Mont., USA). These hypotheses were selected because the linkage studies had been performed on a different population, and hence there was no previous knowledge about linkage or association in the U.S. population described here. The VAS-P score, defined as the summation of the individual's responses in a particular domain, and the VAS-P severity scale, derived using LCCA, were used independently as phenotypes. The significance of markers was determined by using both the raw P-value and a permutation-based approach with B=10,000 permutations.

Interaction Effects on the Severity of Symptoms:

A generalized Cochran-Mantel-Haenszel (CMH) test was implemented in R. R Development Core Team, R: A language and environment for statistical computing, Vienna: R Foundation for Statistical Computing (2010). For all possible pairs of markers located on different chromosomes and significantly associated either with the VAS-P score or the severity of symptoms, the CMH test was used to determine whether pairs of SNPs were independent, conditioned on the levels of the severity phenotype (severe and not severe) defined according to the severity determination described above. This approach was stratified by domains and a total of p pairs of markers were found to be of potential interest. By domain, the False Discovery Rate (FDR) was used to correct by multiple testing. Benjamini et al., Journal of the Royal Statistical Society Series B (Methodological), 57 (1): 289-300 (1995).

To perform the CMH test for a particular domain we used the severity scale as the stratum indicator, such that a series ofp pairs of contingency tables, each of dimension r×c, can be obtained. In this context, p (p=587) is the number of possible pairs of markers located within different chromosomes, r (r=3) is the number of possible genotypes in marker 1, and c (c=3) the number of possible genotypes in marker 2. To avoid any bias inherent to family data, only CMH testing used only those families having the following criteria: (i) discordant parents and concordant children affected, (ii) two discordant siblings and unaffected parents, (iii) unaffected parents and several discordant children and (iv) discordant parents and discordant siblings. Families falling in the third case were further examined so only one affected and one unaffected child was selected. This selection was performed matching by gender and age range. Parents were excluded from families falling in the fourth case. At the end of the process, only one discordant pair was selected within those families that fulfilled the inclusion criteria.

Severity of Symptoms of ADHD and Associated Disorders:

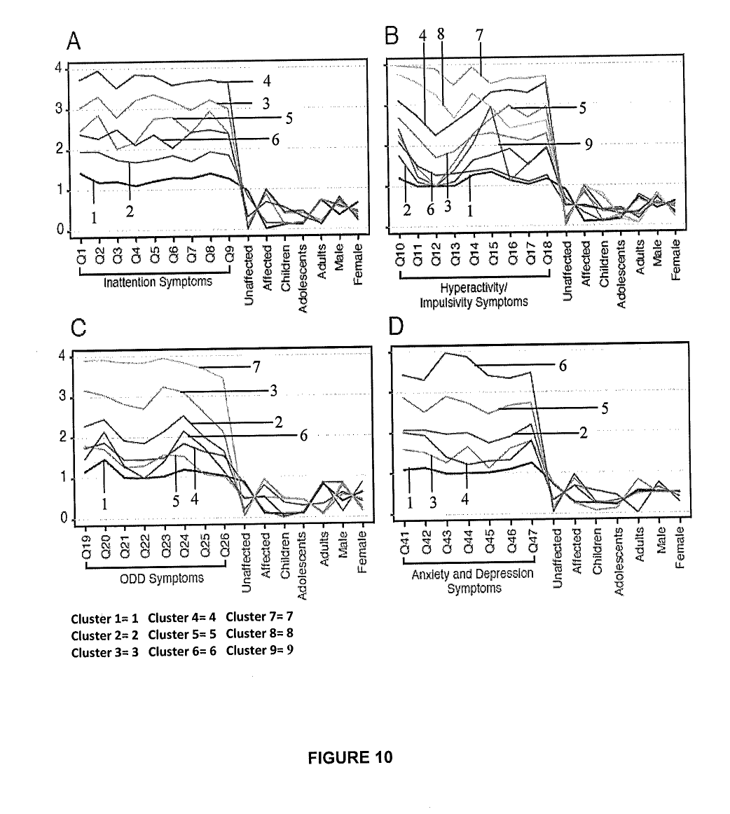

In the maximally expanded sample (n=1,341) the following were identified: six significant independent and mutually exclusive clusters for the VAS-P Inattention domain, nine for Hyperactivity/Impulsivity domain, seven for ODD domain, and six for the A/D domain. See Table 2 and FIGS. 10A-10D. These were consistent with findings from previous studies. Acosta et al., J. Am. Acad. Child Adolesc. Psychiatry 47(7): 797-807 (2008); Arcos-Burgos et al., Mol. Psychiatry; 15(11): 1053-66 (2010); Hudziak et al., J Am Acad Child Adolesc Psychiatry, 37(8): 848-57, (1998), Rohde et al., J. Am. Acad. Child Adolesc. Psychiatry, 40(6): 711-8 (2001), Jain et al., Biol Psychiatry. 61(12): 1329-39 (2007), Elia et al., Psychiatry Res., 170(2-3): 192-8 (2009). Because of small variability in the predictors in the CD domain, LCCA models could not be fitted to this data. Individuals were considered as having severe symptoms when belonging to clusters 3, 4, 5, and 6 in the Inattention domain (n=647, 49%); to clusters 3, 4, 5, 7 and 8 in the Hyperactivity/Impulsivity domain (n=532, 39.7%); to clusters 3 and 7 in the ODD domain (n=229, 17%) and to clusters 5 and 6 in the A/D domain (n=177, 13.2%) as indicated in FIGS. 10A-10D. Under this categorization, severe versus not severe status involves all of the independent variation conferred by the spectrum of symptoms that define ADHD and its comorbidities. Further, using this categorization for each domain, association and interaction analyses was performed for the severity phenotype. Because severity was analyzed by two methods (by overall VAS-P score and by quantifying the magnitude of inattention, hyperactivity/impulsivity, CD, ODD and A/D symptoms using LCC), the severity determination included all of the clinical information from the individuals regardless of their categorically diagnosed status. This approach to analyzing severity is well suited for use in a clinical context, where severity of symptoms is a major predictor of functional outcome.

TABLE 2

| | | | Characteristic and | Predominant | | |
|---|---|---|---|---|---|---|
| | | | Affection Status | Gender and | Severity of | |
| Questions | Criteria | Cluster | for Broader Phenotype | Age Classes | Symptoms | n (%) |

Inattention

| | | | | | | | |
|---|---|---|---|---|---|---|---|
| 1-9 | >5 | 1 | Minimal symptoms, unaffected | Females- | Not severe | 370 | (27.6) |
| | | | for ADHD | Adults | | | |
| | | 2 | Few symptoms, unaffected for | Males-Adults | Not severe | 313 | (23.4) |
| | | | ADHD | | | | |
| | | 3 | Mostly inattentive, high | Males- | Severe | 271 | (20.2) |
| | | | presence of symptoms, | Children & | | | |
| | | | affected for ADHD | Adolescents | | | |
| | | | | (few Adults) | | | |
| | | 4 | Mostly inattentive, higher | Males- | Severe | 182 | (13.6) |
| | | | presence of symptoms, | Children & | | | |
| | | | affected for ADHD | adolescents | | | |
| | | | | (few Adults) | | | |
| | | 5 | Fewer symptoms, affected for | Males-Adults | Severe | 151 | (11.3) |
| | | | ADHD | | | | |
| | | 6 | Fewer symptoms, affected for | Males- | Severe | 53 | (4.0) |
| | | | ADHD | Children | | | |

Hyperactivity/Impulsivity

| | | | | | | | |
|---|---|---|---|---|---|---|---|
| 10-18 | >5 | 1 | Minimal H/I symptoms, most | Females- | Not severe | 418 | (31.2) |
| | | | individuals unaffected for | Adults | | | |
| | | | ADHD | | | | |
| | | 2 | Minimal H/I symptoms, most | Males-Adults | Not severe | 307 | (22.9) |
| | | | individuals unaffected for | | | | |
| | | | ADHD | | | | |
| | | 3 | Few symptoms, affected for | Males- | Severe | 165 | (12.3) |
| | | | ADHD | Adolescents | | | |
| | | 4 | High presence of symptoms | Males- | Severe | 165 | (12.3) |
| | | | except for Q12, most | Children & | | | |
| | | | individuals affected for ADHD | Adolescents | | | |
| | | | | (few Adults) | | | |
| | | 5 | Few symptoms for Q10-14 and | Females- | Severe | 101 | (7.5) |
| | | | fewer for Q15-Q18, | Adults | | | |
| | | | individuals are mostly affected | | | | |
| | | 6 | Few symptoms for Q10 and | Males-All | Not severe | 58 | (4.3) |
| | | | Q11, but minimal symptoms | Ages | | | |
| | | | for the rest; ADHD affection | | | | |
| | | | status is equally present | | | | |
| | | 7 | Mostly H/I individuals, all | Males- | Severe | 53 | (4.0) |
| | | | affected for ADHD; higher | Children | | | |
| | | | presence of symptoms | | | | |
| | | 8 | Mostly H/I individuals affected | Males- | Severe | 48 | (3.6) |
| | | | for ADHD; high presence of | Children | | | |
| | | | symptoms | | | | |
| | | 9 | Few-to-minimal symptoms | Males- | Not severe | 25 | (1.9) |
| | | | except in Q15; ADHD | Children | | | |
| | | | affection status is equally | | | | |
| | | | present | | | | |

ODD

| | | | | | | | |
|---|---|---|---|---|---|---|---|
| 19-26 | >3 | 1 | Minimal symptoms, most | Female- | Not severe | 365 | (27.2) |
| | | | individuals unaffected for | Adults | | | |
| | | | ADHD | | | | |
| | | 2 | Few symptoms, most | Male-All | Not severe | 293 | (21.9) |
| | | | individuals affected for ADHD | Ages | | | |
| | | 3 | High presence of symptoms in | Males- | Severe | 182 | (13.6) |
| | | | Q19-25 and few for Q26; most | Children | | | |
| | | | individuals affected for ADHD | | | | |
| | | 4 | Minimal symptoms in all | Males-Adults | Not severe | 161 | (12) |
| | | | questions; individuals are | | | | |
| | | | mostly unaffected for ADHD | | | | |
| | | 5 | Minimal symptoms in all | Males- | Not severe | 149 | (11.1) |
| | | | questions; ADHD affection | Children & | | | |
| | | | status is equally present | Adolescents | | | |
| | | | | (few Adults) | | | |
| | | 6 | Few symptoms in Q20 and | Females- | Not severe | 143 | (10.7) |
| | | | Q24; ADHD affection status is | Adults | | | |
| | | | equally present | | | | |
| | | 7 | Higher presence of symptoms | Males- | Severe | 47 | (3.5) |
| | | | in all questions; most | Children & | | | |
| | | | individuals affected for ADHD | Adolescents | | | |
| | | | | (few Adults) | | | |

Anxiety and Depression

| | | | | | | | |
|---|---|---|---|---|---|---|---|
| 41-47 | >2 | 1 | Minimal symptoms in all | Males-Adults | Not severe | 375 | (28.0) |
| | | | questions; individuals are | | | | |
| | | | mostly unaffected for ADHD | | | | |
| | | 2 | Fewer symptoms; mostly | Females- | Not severe | 376 | (28.1) |
| | | | affected individuals | Adults | | | |
| | | 3 | Few symptoms; most | Males- | Not severe | 255 | (19) |
| | | | individuals unaffected for | Children | | | |
| | | | ADHD | | | | |
| | | 4 | Fewer symptoms in Q41, Q42 | Males- | Not severe | 157 | (11.7) |
| | | | and Q47 with few on the rest; | Children | | | |
| | | | most individuals affected for | | | | |
| | | | ADHD | | | | |
| | | 5 | High presence of symptoms; | Females- | Severe | 132 | (9.9) |
| | | | most individuals affected for | Adults | | | |
| | | | ADHD | | | | |
| | | 6 | Higher presence of symptoms | Females- | Severe | 45 | (3.4) |
| | | | in all questions; most | Adults | | | |
| | | | individuals affected for ADHD | | | | |

Having identified the severity of symptoms for each of the four VAS-P domains, a Family-Based Association Test (FBAT) was performed, taking into account the complete family structure and the severity status (severe and not severe) as disease indicator (outcome). In a complementary analysis, the overall VAS-P score for each dimension was also used as the outcome for the FBAT as a general indicator of severity.

Table 3 presents the FBAT results for both the LCCA-derived severity status and the overall VAS-P score for each domain. After permutation, a total of thirty-eight genetic markers were found to be significantly associated either with the VAS-P score or the severity of symptoms by dimension.

TABLE 3

| | | | | | Score | | | | | Severity | | | |
|---|---|---|---|---|---|---|---|---|---|---|---|---|---|
| | | | | | In- | | | | | In- | | | |
| | | Al- | | Ef- | atten- | | | | | atten- | | | |
| Marker | Gene | lele | Freq. | fect | tion | H/I | ODD | CD | A/D | tion | H/I | ODD | A/D |

Chromosome 4

| | | | | | | | | | |
|---|---|---|---|---|---|---|---|---|---|
| rs1947275 | LPHN3 | C | 0.814 | + | 0.00819** | | | | |
| rs2132074 | LPHN3 | G | 0.618 | + | | 0.0017** | | | |
| rs4552500 | LPHN3 | G | 0.608 | + | | 0.0031* | | | |
| rs13124636 | LPHN3 | G | 0.046 | − | | 0.0414* | | | |
| rs4860091 | LPHN3 | T | 0.386 | + | | | 0.0054* | | |
| rs335322 | LPHN3 | G | 0.575 | + | | | 0.007* | | |
| rs10015239 | LPHN3 | A | 0.577 | + | | | 0.0098* | | |
| rs35106420 | LPHN3 | G | 0.985 | + | | | | 0.0154* | |
| rs12646895 | LPHN3 | G | 0.508 | + | | | | 0.0019* | |
| rs1510920 | LPHN3 | C | 0.061 | + | | | | 0.0368* | |
| rs186750 | LPHN3 | A | 0.243 | + | | | | 0.0216* | |
| rs734644 | LPHN3 | T | 0.271 | + | | | | | 0.0117* |
| rs6813183 | LPHN3 | G | 0.300 | + | | | | | 0.0114* |
| rs6551670 | LPHN3 | A | 0.300 | + | | | | | 0.0105* |
| rs6551669 | LPHN3 | C | 0.300 | + | | | | | 0.0105* |

Chromosome 5

| | | | | | | | | |
|---|---|---|---|---|---|---|---|---|
| rs249637 | NDFIP1 | G | 0.074 | − | 0.0216* | | | |
| rs37022 | SLC6A3 | T | 0.831 | + | | 0.0052** | | |
| rs250682 | SLC6A3 | C | 0.798 | + | | 0.0082* | | |
| rs6596271 | SLC25A48 | G | 0.042 | − | | | 0.0052* | |
| rs164080 | GNPDA1 | C | 0.526 | + | | | | 0.0015* |

Chromosome 11

| | | | | | | | | | | | | |
|---|---|---|---|---|---|---|---|---|---|---|---|---|
| rs10891551 | DRD2 | A | 0.122 | + | 0.0253* | | | | | | | |
| rs719804 | TTC12 | G | 0.223 | − | 0.0117* | | | | | | | |
| rs4938006 | NCAM1/ | G | 0.109 | + | | 0.034* | 0.0106** | | | | | 0.0359**^{,1} |
| | TTC12 | | | | | | | | | | | |
| rs12799083 | DRD2 | C | 0.031 | − | | | 0.0107* | | | | | |
| rs4245148 | DRD2 | T | 0.122 | + | | | 0.0251* | | | | | |
| rs17596017 | NCAM1 | T | 0.024 | − | | | | 0.0191* | | | | |
| rs1381246 | BSX | C | 0.451 | − | | | | 0.0057* | | | | |
| rs652285 | NCAM1 | T | 0.057 | + | | | | | 0.0097* | 0.0082* | | 0.0214* |
| rs675646 | NCAM1 | C | 0.071 | + | | | | | 0.0107* | 0.0807* | | 0.0105** |
| rs649568 | NCAM1 | T | 0.044 | + | | | | | 0.0171* | | | 0.0162* |
| rs702966 | PHRF1 | C | 0.276 | − | | | | | 0.0121* | | | |
| rs2574829 | NCAM1 | G | 0.394 | + | | | | | | 0.0084* | | |
| rs11214521 | NCAM1 | G | 0.035 | − | | | | | | | 0.0226 | |
| rs11214505 | NCAM1 | G | 0.082 | − | | | | | | | 0.0292 | |
| rs1055076 | TTC12 | A | 0.175 | + | | | | | | | 0.0343 | |
| rs12222469 | NCAM1 | A | 0.035 | + | | | | | | | | 0.0183* |

Chromosome 12

| | | | | | |
|---|---|---|---|---|---|
| rs17722134 | TPH2 | G | 0.029 | − | 0.0085* |

Chromosome 17

| | | | | | |
|---|---|---|---|---|---|
| rs720S257 | ARRB2 | C | 0.055 | + | 0.0164* |

¹Effect on the severity towards opposite direction to the one described in the “Effect” column
*Permutated P-value < 0.05
**Permutated P-value < 0.01
H/I: Hyperactivity/Impulsivity; ODD: Oppositional Defiant Disorder; CD: Conduct Disorder.
Not significant P-values after B = 10,000 permutations are shown in italic.
No correction for the number of questions interrogated was performed in genetic association analyses

In the Inattention domain, a total of eight markers were found to be associated with either the LCCA-derived severity status or the VAS-P score. Using the VAS-P score, significantly associated markers identified were linked to LPHN3 (4q), NDFIP1 (5q), and DRD2 (11q) and TTC12 (11q). Using the severity of symptoms, all significantly associated markers were linked to NCAM1 (11q).

For the Hyperactivity/Impulsivity domain, two markers linked to SLC6A3 (5p) were found to be associated with the VAS-P score, and three markers linked to NCAM1 (11q) were associated with the severity of symptoms.

For the ODD domain, four markers were found to be associated with the VAS-P score and four with the severity of symptoms. Using the VAS-P score, three of the four markers identified were linked to LPHN3 (4q), and one was linked to NCAM1 (11q). Using severity of symptoms, all significant markers were linked to LPHN3 (4q).

For CD, six markers were found to be associated with the VAS-P score, three were linked to LPHN3 (4q) and three were linked to NCAM1 (11q).

In the A/D domain, a total of eight markers were found to be associated with VAS-P score and six with severity of symptoms. Among the markers found to be associated with VAS-P score, four were linked to LPHN3 (4q), two were linked to NCAM1 (11q), one was linked to GNPDAI (5), and one was linked to TPH2 (12q). Using severity of symptoms, a total of six markers were found to be associated, five were linked to NCAM1 (11q), one was linked to GNPDAI (5), and is located at an intergenic region between NCAM1 and TTC12 on chromosome 11.

This foregoing results show that genetic markers, including markers linked to LPHN3 (4q), NDFIP1 (5q), DRD2 (11q), TTC12 (11q), TPH2 (12q), SLC6A3 (5p), GNPDA1 (5), and NCAM1 (11q) can be used to determine or predict the severity of symptoms of ADHD and associated disorders in a subject.

### Example 6

This example demonstrates that an LPHN3-11q two-locus cooperative interaction is a predictor of the severity of symptoms of ADHD and associated disorders.

By domain, a total of five hundred and sixty-seven unique pairs of markers located at different chromosomes were tested for SNP×SNP interactions. After breaking up the family data obtained using the population and methods described in Example 5, the number of severe (cases)/not severe (controls) pairs of individuals available for analysis was found to be one hundred and eighty-four for the Inattention domain, one hundred and sixty-nine for the Hyperactivity/Impulsivity domain, one hundred and four for the ODD domain, and seventy-six for the A/D domain. The results of CMH tests after correcting by multiple testing using FDR are shown in FIG. 11. Regardless of the VAS-P domain being considered, statistically significant SNP×SNP interactions included markers within LPHN3 and within the region of chromosome 11 containing NCAM1, DRD2 and TTC12 genes. See Table 3 and FIG. 11.

For the Inattention domain, the interaction between rs1947275 and rs17596017 was found to be statistically significant (M²=33.163, P_{FDR-corrected}=0.00067). Comparison of the genotype distributions for these markers between cases and controls showed that decreasing the number of copies of the T allele in rs 1947275 marker within LPHN3 (from CT to CC) yields a reduction of the number double homozygous individuals for the C allele of marker rs17596017 linked to NCAM1 (11q). See FIG. 11.

For the Hyperactivity/Impulsivity domain, in which the interaction between rs35106420 and rs620291 was significant (M²=20.497, P_{FDR-corrected}=0.02766), similar results were obtained. For example, when the A allele is present in rs35106420 in LPHN3 and the C allele is present in rs620291 linked to NCAM1 (11q) (while keeping the other fixed), the number of cases decreases when compared with the controls. See FIG. 11E.

For ODD, two interactions were found to be statistically significant, one including markers rs995447 and rs11214505 (M²=41.379, P_{FDR-corrected}=0.00008), and another including markers rs734644 and rs620291 (M²=26.795, P_{FDR-corrected}=0.00196). In the former interaction, the presence of one copy of the G allele in NCAM1 yields a reduction in the number of cases with two copies of the C allele in LPHN3 and an increment of those with only one copy of it. In the latter, regardless the number of copies of the A allele in LPHN3, the number of cases with one or more copies of the T allele in NCAM1 is greater than the number of controls. See FIG. 11.

In the A/D domain, the interaction between rs1510920 and rs4938006 was statistically significant (M²=23.973, P_{FDR-corrected}=0.00655). For instance, when the one copy of the G allele is present in NCAM1, it yields a reduction in the number of cases with one or more copies of the A allele in LPHN3. See FIG. 11.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of determining whether a subject has an increased susceptibility to develop Attention Deficit Hyperactivity Disorder (ADHD), the method comprising:
a) obtaining a sample from a subject;
b) analyzing the sample for the presence one or more ADHD susceptibility markers in the latrophilin 3 gene (LPHN3) located in the region spanning positions 61,650,000-62,650,000 in chromosome 4q;
c) analyzing the sample for the presence of one or more ADHD susceptibility markers located in the region spanning positions 112,250,000-112,900,000 in chromosome 11q; and
d) determining whether the subject has a susceptibility to develop ADHD, wherein the presence of the one or more ADHD susceptibility markers in LPHN3 and the presence of one or more ADHD susceptibility markers in chromosome 11q indicate an increased susceptibility to develop ADHD.

2. The method of claim 1, wherein the method comprises detecting nucleic acids in the sample with sequences comprising (i) at least one ADHD susceptibility marker located in an approximately 240 kb region of chromosome 4q within LPHN3 and (ii) at least one ADHD susceptibility marker in a 166 kbps region of chromosome 11q which spans from intron 7 of the gene coding neural cell adhesion molecule 1 (NCAM1), encompasses the tetratricopeptide repeat domain 12 gene (TTC12) and the ankyrin repeat and kinase domain containing 1 gene (ANKK1), and is adjacent to the 5′ UTR of the dopamine receptor D2 gene (DRD2).

3. The method of claim 1, wherein the method comprises analyzing the sample for nucleic acid comprising at least one ADHD susceptibility marker in LPHN3 selected from the group consisting of the rs7678046, rs1901223, rs6813183, and rs1355368 polymorphisms (in SEQ ID NO:1 or its complement) associated with increased ADHD susceptibility.

4. The method of claim 1, wherein the method comprises analyzing the sample for nucleic acid comprising at least one ADHD susceptibility marker in LPHN3 selected from the group consisting of the rs1038426 (in SEQ ID NO:4 or its complement), rs6551665 (in SEQ ID NO:5 or its complement), rs2345039 (in SEQ ID NO:6 or its complement), rs1947274 (in SEQ ID NO:7 or its complement) rs1947275 (in SEQ ID NO:8 or its complement), rs35106420 (in SEQ ID NO:9 or its complement), rs995447 (in SEQ ID NO:10 or its complement), rs1510920 (in SEQ ID NO:11 or its complement), rs2132074 (in SEQ ID NO:12 or its complement), rs4552500 (SEQ ID NO:13 or its complement) polymorphisms associated with increased ADHD susceptibility.

5. The method of claim 1, wherein the method comprises analyzing the sample for one or more ADHD susceptibility markers in chromosome 11q selected from the group consisting of the rs1293344 (in SEQ ID NO:24 or its complement), rs677642 (in SEQ ID NO:25 or its complement), rs877137 (in SEQ ID NO:26 or its complement), rs754672 (in SEQ ID NO:27 or its complement), rs965560 (in SEQ ID NO:28 or its complement) rs17596017 (in SEQ ID NO:29 or its complement), rs620291 (in SEQ ID NO:31 or its complement), rs11214505 (in SEQ ID NO:32 or its complement), rs677642 (in SEQ ID NO:25 or its complement) and rs877137 (in SEQ ID NO:26 or its complement) polymorphisms associated with increased ADHD susceptibility.

6. The method of claim 1, wherein the sample is tissue or blood.

7. The method of claim 1, wherein the method comprises determining the number of allele copies of one or more of the ADHD susceptibility markers analyzed in the sample.

8. A method of predicting or determining the severity of one or more symptoms of Attention Deficit Hyperactivity Disorder (ADHD) or associated disorders in a subject, the method comprising:
a) obtaining a sample from a subject;
b) analyzing the sample for one or more ADHD susceptibility markers or protective markers, wherein the one or more markers are linked to the NDFIP1 gene on chromosome 5q, the GNPDA1 gene on chromosome 5q, the NCAM1 gene on chromosome 11q, or the intergenic region between NCAM1 and TTC12 on chromosome 11q; and
c) predicting or determining the severity of one or more symptoms of ADHD or an associated disorder in the subject, wherein the presence of the one or more ADHD susceptibility or protective markers indicate the severity of one or more symptoms of ADHD or an associated disorder.

9. The method of claim 8, wherein the method comprises predicting or determining the severity of inattention symptoms and the method comprises:
b) analyzing the sample for one or more ADHD susceptibility markers linked to the NDFIP1 gene on chromosome 5q, the TTC12 gene on chromosome 11q, or the NCAM1 gene on chromosome 11q; and
c) predicting or determining the severity of inattention in the subject, based on the presence of the one or more ADHD susceptibility markers.

10. The method of claim 8, wherein method comprises predicting or determining the severity of hyperactivity/impulsivity symptoms and the method comprises:
b) analyzing the sample for one or more ADHD susceptibility markers linked to the NCAM1 gene on chromosome 11q; and
c) predicting or determining the severity of hyperactivity/impulsivity in the subject based on the presence of the one or more ADHD susceptibility markers.

11. The method of claim 8, wherein method comprises predicting or determining the severity of oppositional defiant disorder (ODD) and the method comprises:
b) analyzing the sample for one or more ADHD susceptibility markers linked to the NCAM1 gene on chromosome 11q; and
c) predicting or determining the severity of ODD in the subject based on the presence of the one or more ADHD susceptibility markers.

12. The method of claim 8, wherein method comprises predicting or determining the severity of conduct disorder and the method comprises:
b) analyzing the sample for one or more ADHD susceptibility markers linked to the NCAM1 gene on chromosome 11q; and
c) predicting or determining the severity of conduct disorder in the subject based on the presence of the one or more ADHD susceptibility markers.

13. A method of predicting or determining the severity of one or more symptoms of Attention Deficit Hyperactivity Disorder (ADHD) or associated disorders in a subject, the method comprising:
a) obtaining a sample from the subject;
b) analyzing the sample for one or more ADHD susceptibility or protective markers in LPHN3 located in the region spanning position 61,650,000-62,650,000 on chromosome 4q;
c) analyzing the sample for one or more ADHD susceptibility or protective markers located in the region spanning position 112,250,000-112,900,000 of chromosome 11q; and
d) predicting or determining the severity of one or more symptoms of ADHD or an associated disorder in the subject based on the presence of the one or more ADHD susceptibility or protective markers on chromosome 4q and the one or more ADHD susceptibility or protective markers on chromosome 11q.

14. The method of claim 13, wherein analyzing the sample for one or more ADHD susceptibility markers comprises analyzing the sample for one or more ADHD susceptibility markers linked to the DRD2 gene, the NCAM1 gene, or the TTC12 gene on chromosome 11q.

15. The method of claim 13, wherein analyzing the sample for one or more ADHD susceptibility markers in chromosome 11q, comprises detecting nucleic acid in the sample with a sequence comprising at least one marker selected from the group consisting of the rs10891551, rs719804, rs4938006, rs12799083, rs4245148, rs17596017, rs1381246, rs652285, rs675646, rs649568, rs702966, rs2574829, rs11214521, rs11214505, rs1055076, and rs12222469 polymorphisms which indicate the severity of the one or more ADHD symptoms.

16. The method of claim 13, wherein method comprises predicting or determining the severity of inattention symptoms in the subject and the method comprises:
b) analyzing the sample for the ADHD susceptibility marker rs1947275; and
c) analyzing the sample for one or more of the ADHD susceptibility markers rs17596017 or rs12799083,
d) predicting or determining the severity of inattention symptoms in the subject, wherein 0 or 1 copies of the T allele in rs1947275, and 2 copies of the C allele in rs17596017 or 2 copies of the G allele in rs12799083, indicate severe inattention symptoms and wherein 1 or 2 copies of the T allele in rs17596017 or 1 or 2 copies of the C allele in rs12799083 indicate less severe inattention symptoms.

17. The method of claim 13, wherein method comprises predicting or determining the severity of hyperactivity/impulsivity symptoms in the subject and the method comprises:
b) analyzing the sample for the ADHD susceptibility marker rs35106420; and
c) analyzing the sample for the ADHD susceptibility marker rs620291,
wherein two copies of the G allele in rs35106420 and 1 or 2 copies of the C allele in rs620291 indicate severe hyperactivity/impulsivity symptoms and wherein the presence of the A allele in rs35106420 and two copies of the G allele in rs620291 indicate less severe hyperactivity/impulsivity symptoms.

18. The method of claim 13, wherein method comprises predicting or determining the severity of oppositional defiant disorder (ODD) in the subject and the method comprises:
b) analyzing the sample for the ADHD susceptibility marker rs995447; and
c) analyzing the sample for the ADHD susceptibility marker rs11214505,
wherein two copies of the T allele in rs995447 and 1 or 2 copies of the A allele of rs11214505 indicate severe ODD symptoms and wherein 1 copy of the C allele in rs995447 and two copies of the G allele of rs11214505 indicate less severe ODD symptoms.

19. The method of claim 13, wherein method comprises predicting or determining the severity of ODD in the subject and the method comprises:
b) analyzing the sample for the ADHD susceptibility marker rs734644; and
c) analyzing the sample for the ADHD susceptibility marker rs620291,
wherein 1 or 2 copies of the C allele in rs734644 and 1 or 2 copies of the C allele of rs620291 indicate severe ODD symptoms and wherein 2 copies of the T allele in rs734644 and one or two copies of the A allele of rs620291 indicate less severe ODD symptoms.

20. The method of claim 13, wherein method comprises predicting or determining the severity of anxiety and depression in the subject and the method comprises:
b) analyzing the sample for the ADHD susceptibility marker rs 1510920; and
c) analyzing the sample for the ADHD susceptibility marker rs4938006,
wherein 1 or 2 copies of the A allele in rs1510920 and 1 or 2 copies of the A allele of rs4938006 indicate severe anxiety and depression symptoms, provided that the sample is not heterozygous for both rs1510920 and rs4938006, and wherein 2 copies of the T allele in rs1510920 and 2 copies of the G allele of rs4938006 indicate less severe anxiety and depression symptoms.

21. The method of any one of claim 13, wherein the sample is a tissue or blood.

22. A method of monitoring a subject with an altered susceptibility to develop ADHD, the method comprising:
1) determining whether a subject has an increased susceptibility to develop ADHD according to the method of claim 1, and
2) monitoring the subject for development of ADHD or worsening symptoms of ADHD.

23. A method of treating one or more symptoms of ADHD or associated disorders in a subject, the method comprising:
1) predicting or determining the severity of one or more symptoms of ADHD or an associated disorder in the subject according to the method of claim 8, and
2) providing the subject with treatment appropriate to the severity of the one or more symptoms of ADHD or an associated disorder, wherein the treatment is appropriate for a subject that is predicted or determined to have the severe form of the one or more symptoms of ADHD or an associated disorder.

24. The method of claim 23, wherein the subject has is predicted or determined to have a severe form of one or more symptoms of ADHD or an associated disorder and the method includes providing pharmacological therapy comprising a stimulant.

25. The method of claim 23, wherein the subject is predicted or determined to have increased severity of anxiety and depression and the method includes providing pharmacological therapy that comprises an antidepressant.
